(19) United States
(12) Patent Application Publication
(10) Pub. No.: US 2003/0170689 A1
(43) Pub. Date: Sep. 11, 2003

(54) DNA microarrays comprising active chromatin elements and comprehensive profiling therewith

(75) Inventors: John A. Stamatoyannapoulos, Boston, MA (US); Michael McArthur, Rockland (GB)

Correspondence Address: Pennie & Edmonds LLP, 1155 Avenue of the Americas, New York, NY, 10036-2711, US

(73) Assignee: Rexagen Corporation, Seattle, WA (US)

(21) Appl. No.: 10/319,440

(22) Filed: Dec. 12, 2002

Related U.S. Application Data
(63) Continuation-in-part of application No. PCT/US02/15032, filed on May 13, 2002.
(60) Provisional application No. 60/290,036, filed on May 11, 2001.

(51) Int. Cl. C12Q 1/68; G06F 19/00; G01N 33/48; G01N 33/50; C12M 1/34

(52) U.S. Cl. 435/6; 435/287.2; 702/20

(57) ABSTRACT

Arrays, probes and methods are disclosed for the construction and interrogation of DNA arrays containing Active Chromatin Elements, and thereby active genetic regulatory sequences. Further methods are disclosed for interrogation of such arrays in order to reveal the pattern of genetic regulatory activity within any given cell or tissue type or associated with any particular genetic locus under a variety of conditions.

### FIELD OF THE INVENTION

The invention relates to DNA arrays for simultaneous detection of multiple nucleic acid sequences, their manufacture and use. The invention further concerns array methods and devices for detecting patterns of active chromatin elements, and particularly genetic control elements active in eukaryotic cells.

### BACKGROUND OF THE INVENTION

Conventional gene expression studies generally employ immobilized DNA molecules that are complementary to gene transcripts (either the entire transcript or to selected regions thereof) that are transcribed and spliced into mRNA. Recent advances in this field utilize arrays or microarrays of such molecules that enable simultaneous monitoring of multiple distinct transcripts (see, e.g., Schena et al., Science 270:467-470 (1995); Lockhart et al., Nature Biotechnology 14:1675-1680 (1996); Blanchard et al., Nature Biotechnology 14, 1649 (1996); and U.S. Pat. No. 5,569,588, issued Oct. 29, 1996 to Ashby et al. entitled “Methods for Drug Screening.”). Such arrays have the potential to detect transcripts from virtually all actively transcribed regions of a cell or cell population, provided the availability of an organism's complete genomic sequence, or at least a sequence or library comprising all of its gene transcripts. In the case of the Human where a complete gene set remains unclear, such arrays may be employed to monitor simultaneously large numbers of expressed genes within a given cell population.

The simultaneous monitoring technologies particularly relate to identifying genes implicated in disease and in identifying drug targets (see, e.g., U.S. Pat. Nos. 6,165,709; 6,218,122; 5,811,231; 6,203,987; and 5,569,588). Unfortunately, these array technologies generally rely on direct detection of expressed genes and therefore reveal only indirectly the activity of genetic regulatory pathways that control gene expression itself. On the other hand, a detection system directed toward sensing the activity of particular genetic regulatory pathways or cis-acting regulatory elements could provide deeper information concerning a cell's regulatory state. Accordingly, the detection of active regulatory elements, particularly in related and interacting groups, potentially could become extremely important for delineation of regulatory pathways, and provide critical knowledge for design and discovery of disease diagnostics and therapeutics.

Most research in the area of gene regulation has focused on finding and using individual sequences either upstream or downstream of individual coding gene targets. Generally, the presence of absence of a particular DNA sequence is linked with increased or decreased expression of a nearby gene when determining the regulatory effect of the sequence. For example, the beta-like globin gene was shown to contain four major DNase I hypersensitive sites of possible regulatory function by studies that removed or added these sequences and that looked for an effect on gene expression in erythroid cells. See Grosveld et. al. U.S. Pat. No. 5,532,143. From related studies, Townes et al. asserted that two of the four DNAse hypersensitive sites might control genes generally in cells of erythroid lineage. Although an interesting development, these observations generally are limited to detection of effects on nearby coding sequences of known genes. Multiple regulatory units, which behave coordinately, are not readily amenable to analysis by these techniques.

Multiple gene and protein elements interact for even simple biological processes. Because of this, a one at a time strategy for targeting a single coding gene and nearby non-coding sequences to determine their effects on the preselected gene insufficiently addresses the true in vivo situation. Accordingly, any tool that can provide simultaneous regulation system information would give rich benefits in terms of improved diagnosis, clinical treatment and drug discovery.

### SUMMARY OF THE INVENTION

The present invention overcomes the problems and disadvantages associated with current strategies and designs with methods and materials that enable the use of nucleic acid arrays for profiling large numbers of active chromatin elements (‘ACE’), and hence active genetic regulatory units.

One embodiment of the invention is directed to methods for manufacturing an array of genomic regulatory elements. Since virtually all active genomic regulatory regions are contained within ACEs, an array of ACEs constitutes an array of regulatory elements. Generally, a nucleic acid microarray is made having spots that contain copies of sequences corresponding to a genomic DNA sequence that contains an ACE or a putative genomic regulatory element. In certain illustrative embodiments, the nucleic acid sequences are obtained by amplifying sequences from a library, e.g., a library of ACE sequences as described herein, using the polymerase chain reaction, and depositing material with a microarraying apparatus, or synthesizing ex situ using an oligonucleotide synthesis device, and subsequently depositing using a microarraying apparatus, or synthesizing in situ on the microarray using a method such as piezoelectric deposition of nucleotides.

Another embodiment of the invention is directed to methods for analyzing ACEs comprising: preparing chromatin from a target cell population; treating said chromatin with an agent that induces modifications at hypersensitive sites in chromatin such as a non-specific restriction endonuclease to induce single and double stranded cleavage at such locations in marked preference to other locations within the genome; modifying the fragment ends through the ligation of a linker adapter or similar means to tag the sequences in a manner such that they can be separated from the mixture; modifying the fragments to reduce the average fragment size by digest with a restriction enzyme or by sonication or an equivalent procedure; labeling the fragment subpopulation containing hypersensitive site sequences with a fluorescent dye or other marker sufficient for detection through an automated apparatus such as a DNA microarray reader; incubating the labeled fragment population with a microarray according to the present invention and recording the signal intensity at each array coordinate. In this way, one can effectively and efficiently identify a collection of ACEs associated with. e.g., active within, the sample from which the labeled fragment population was derived.

Yet another embodiment of the invention is a procedure for profiling ACEs from an organism, comprising a first step of constructing a DNA microarray that contains genomic regulatory elements, and a second step of probing the microarray to assay regulatory element activation. The first step involves constructing a DNA microarray having spots with one or more copies of a DNA sequence corresponding to a genomic DNA sequence that contains a nuclease hypersensitive site or a putative genomic regulatory element. The DNA sequences contained on the array may be obtained or deposited alternative ways, for example: by amplifying the DNA sequences using PCR from a library, such as a nuclease hypersensitive site library, containing such sequences, and subsequently depositing with a microarraying apparatus; synthesizing the DNA sequences ex situ with an oligonucleotide synthesis device, and subsequently depositing with a microarraying apparatus; or by synthesizing the DNA sequences in situ on the microarray by, for example, piezoelectric deposition of nucleotides. The number of sequences deposited on the array may vary between 10 and several million depending on the technology employed to create the array.

In another embodiment of the invention a DNA microarray containing genomic DNA sequences corresponding to established or putative regulatory elements is assayed in five steps. In step one, chromatin from a target cell population is prepared and treated with an agent that induces modifications at ACEs. For example, the non-specific restriction endonuclease DNAse may be used to induce single and double stranded cleavage at such locations in marked preference to other locations within the genome. Secondly, the fragment ends are modified through the ligation of a linker adapter, enzymatic labeling or similar means to tag the sequences in a manner such that they can be separated from the mixture. Thirdly, the DNA fragments may be modified further to reduce the average fragment size by digest with a restriction enzyme, by sonication or an equivalent procedure. Fourthly, the DNA fragment subpopulation containing hypersensitive site sequences is labeled with a fluorescent dye or other marker sufficient for detection through an automated apparatus such as a DNA microarray reader. A last step is incubation of the labeled fragment population with a DNA microarray according to the present invention and recording the signal intensity at each array coordinate.

According to another aspect of the invention there is provided a method of ascertaining the effect of a test compound, e.g., a chemical agent, biological agent or other environmental perturbation, on a regulatory profile of a tissue obtained from a eukaryotic organism. The method generally involves obtaining a first profile for binding between active chromatin elements of the tissue that is unexposed to the test compound or perturbation and a microarray according to the present invention. A second profile is obtained for binding between active chromatin elements of the tissue and a microarray according to the invention. By comparing the first profile with the second profile, the genetic ACE elements that are effected by the perturbation are thereby revealed. Contact with a test compound or perturbation may occur before obtaining the tissue from the organism and may be selected from the illustrative group consisting of an infection of the eukaryotic organism from a microorganism, loss in immune function of the eukaryotic organism, exposure of the tissue to high temperature, exposure of the tissue to low temperature, cancer of the tissue, cancer of another tissue in the eukaryotic organism, irradiation of the tissue, exposure of the tissue to a chemical or other pharmaceutical compound; and aging. Alternatively, contact with a test compound or perturbation may occur after obtaining the tissue from the organism and may be selected from the illustrative group consisting of exposure of the tissue to high temperature, exposure of the tissue to low temperature, irradiation of the tissue, exposure of the tissue to a chemical or other pharmaceutical compound, and aging.

According to another aspect of the invention, there is provided a method of discerning at least one set of co-regulated genes in cells of a eukaryotic organism, comprising obtaining a first profile for binding between active chromatin elements of the tissue under controlled culture conditions; obtaining a second profile for binding between active chromatin elements of the tissue under conditions where a known regulator of at least one of the genes is altered with respect to the controlled culture conditions; and comparing the first profile with the second profile from b) to determine which genetic elements are effected by the alteration of the known regulator. Illustrative regulators include hormones, nutrients, or pharmacologically active chemicals, and the like.

According to another aspect of the invention, there is provided a method for profiling differential regulatory element activation from two populations that contain nucleic acid. This generally involves first obtaining multiple active chromatin elements from a first population and labeling them with a first label and obtaining multiple active chromatin elements from a second population and labeling them with a second label. The ACEs are then hybridized with a DNA microarray of the present invention, preferably containing DNA species in separate locations that match putative or verified regulatory elements, in order to determine the ratio of signals from the first and second labels within the array. This allows for the rapid and efficient identification of differences in ACE activities between two or more sample populations. In one example, one of the populations is an untreated control and the other population is treated by contact with at least one test compound or other perturbation, and the signal ratios obtained provide an indication of gene regulatory activity by the at least one test compound or perturbation.

According to another aspect of the invention, there is provided a method of identifying an ACE profile associated with a disease state, such as cancer, comprising obtaining a first profile or set of profiles for binding between active chromatin elements of a tissue, said first profile or set of profiles being representative of a normal healthy condition. A second profile or set of profiles is also obtained for binding between active chromatin elements of a tissue, said second profile or set of profiles being representative of a disease condition. By comparing the first profile or set of profiles with the second profile or set of profiles, one can readily identify alterations in the activity of one or more ACE elements in the disease condition relative to the normal condition. The invention thus further encompasses a disease associated ACE profile or set of profiles identified according to the above method, as well as methods for diagnosing the presence of a disease condition in a patient, comprising obtaining an ACE profile for a biological sample obtained from a patient suspected of having said disease condition and comparing said ACE profile to a disease associated ACE profile.

In another aspect, the invention provides methods of preparing probes that may be used according to methods of the invention, including methods of screening arrays and methods of profiling cells and ACEs.

In one embodiment, the invention provides a method of preparing fixed length direct monotagged nucleic acids that includes treating genomic DNA with an agent that cleaves DNA, ligating the treated genomic DNA with a blunt or T-tailed linker containing a type IIs restriction endonuclease restriction site, and treating the ligated DNA with a type IIs restriction enzyme. In one particular embodiment, the cleavage is performed using DNase I in the presence of manganese. In a related embodiment, the agent that cleaves DNA is a restriction endonuclease.

In another embodiment, the invention provides a method of preparing fixed length indirect monotagged nucleic acids that includes treating genomic DNA with an agent that cleaves DNA, capturing the treated genomic DNA, treating the captured genomic DNA with a restriction enzyme, ligating the DNA of with a linker comprising a type IIs restriction enzyme site, and treating the ligated DNA with a type II restriction enzyme. In one particular embodiment, the cleavage sites within the genomic DNA are captured following biotinylation or ligation of a biotinylated linker.

A related embodiment of the invention provides a method of profiling ACEs in a cell, comprising preparing fixed length direct monotagged or fixed length indirect monotagged nucleic acids according to the invention and hybridizing the genomic DNA to an array comprising active chromatin element. Such method may further comprise an identification step, such as, for example, detecting hybridized or bound nucleic acids.

Another related embodiment provides method of profiling a cell, comprising preparing genomic DNA according to the method of claim 120 or 122 and hybridizing the genomic DNA to an array comprising a plurality of DNA sequences. This method may also further comprise an identification step, such as, for example, detecting hybridized or bound nucleic acids.Other embodiments and advantages of the invention are set forth in part in the description which follows, and in part, will be obvious from this description, or may be learned from practice of the invention.

### DESCRIPTION OF THE FIGURES

FIG. 1 is an overview of an embodiment for assaying ACE activity using ACE DNA microarrays.

FIG. 2 illustrates an approach for profiling ACE activity using a two-dye system to increase signal-to-noise ratio.

FIG. 3 illustrates an approach for profiling differential ACE representation in two different samples.

FIG. 4 illustrates an approach for the use of ACE arrays to screen drugs and/or small molecule compounds.

FIG. 5 illustrates an approach for identifying a correlation between ACE activity and gene expression obtained by an embodiment of the invention.

FIG. 6 shows the use of an embodiment for controlling quality of conventional expression arrays.

### DETAILED DESCRIPTION OF THE INVENTION

Nuclease hypersensitive sites from chromatin lack protein coding sequences and generally lack highly repetitive sequences. These sequences (hereinafter termed “ACE”) are putative regulatory sites and as such are part of the set of regulatory elements that suffice to control the entire programme of the genome within a cell.

An Active Chromatin Element (‘ACE’) may be defined as a genomic DNA locale which, in the context of nuclear chromatin, serves as a template for the binding of one or more proteins or protein complexes sufficient to produce a focal alteration in the nucleosomal structure. Such ACEs typically, but not exclusively, range from between 16 base pairs to 200 base pairs to up to 1500 base pairs in extent (e.g., J Biol Chem Jul. 20, 2001;276(29):26883-92).

ACE sequences may be identified, manipulated, characterized and/or used according to illustrative methods provided herein below, and, in addition, according to the disclosures of U.S. Ser. No. 09/432,576, filed Nov. 12, 1999 entitled “Production of Nuclease Hypersensitive Site Libraries”; U.S. Ser. No. 60/378,664, filed May. 9, 2002entitled “DNA Microarrays Comprising Regulatory Elements and Comprehensive Profiling Therewith”; U.S. Ser. No. 10/187,887, filed Jul. 3, 2002 entitled “Global Isolation of Functionally Active Genomic Elements”, PCT/US02/16967, filed May. 30, 2002, entitled “Accurate and Efficient Quantification of DNA Sensitivity By Real-Time PCR,” and U. S. Provisional Patent Application “Profiled Regulatory Sites Useful for Gene Control,” filed Dec. 5, 2002.

Identification of ACEs

In one preferred embodiment of the invention, an ACE at a particular genomic locale may be revealed through its differential sensitivity (‘hypersensitivity’) to the action of DNA modifying agents such as, for example, the non-specific endonuclease DNAse (e.g., EMBO J Jan. 3, 1995; 14(1):106-16). However, whereas all DNAse Hypersensitive Sites are, by definition, ACEs, not all ACEs may be detected through a DNAse Hypersensitivity assay.

Thus, in another embodiment of the invention, ACEs may also be revealed through methods which rely on the detection of epigenetic modifications in chromatin such as histone acetylation and cytosine methylation. Treatments which may exert selective effects at ACEs include one or more of the following DNA-modifying agents: nucleases (both sequence-specific and non-specific); topoisomerases; methylases; acetylases; chemicals; pharmaceuticals (e.g., chemotherapy agents); radiation; physical shearing; nutrient deprivation (e.g., folate deprication), etc.

Typically, the identification of ACEs involves the treatment of genomic or chromosomal DNA with an agent that modifies DNA is some manner, such as cleaving one or both strands of DNA. However, there is no requirement that the genomic DNA is isolated or purified prior to treatment. Rather, treatment may be performed on whole cells, and preferably, treatment is performed on isolated nuclei. Thus, the treatment of genomic DNA is preferably performed in the context of chromatin inside a nucleus.

Another embodiment for the identification of ACEs involves modifying the proteins that bind to a given ACE (or set of ACEs) so they induce DNA modification such as strand breakage. Proteins can either be modified by many means, such as incorporation of 1251, the radioactive decay of which would cause strand breakage (e.g., Acta Oncol. 39: 681-685 (2000)), or modifying cross-linking reagents such as 4-azidophenacylbromide (e.g., Proc. Natl. Acad. Sci. USA 89: 10287-10291) which form a cross-link with DNA on exposure to UV-light. Such protein-DNA cross-links can subsequently be converted to a double-stranded DNA break by treatment with piperidine.

Yet another embodiment for the identification of ACEs relies on antibodies raised against specific proteins bound at one or more ACEs, such as transcription factors or architectural chromatin proteins, and used to isolate the DNA from the nucleoprotein complexes associated with ACEs in vivo. An example of a currently used technique cross-links proteins and DNA within the eukaryotic genome following treatment with formaldehyde. After isolation of the chromatin and following either sonication or digestion with nucleases the sequences of interest are immunoprecipitated (Orlando et al. Methods 11: 205-214 (1997)). In one illustrative assay according to this embodiment, the Chromatin Immunoprecipitation (Chip) assay is used for the recovery of DNA sequences from eukaryotic nuclei by antibody recognition of epitopes present on associated proteins within the nucleoprotein complex. This approach can thus be used to recover DNA on the basis of either the enzymatic modifications of the histone proteins (referred to as the histone code and including but not limited to histone H4 and H3 acetylation, histone H3 methylation, histone H1 phosphorylation) or the presence of specific proteins (be they members of the basal transcriptional machinery or certain transcription factors) or post-translationally modified versions of such proteins (which can be modified in a similar way to histone proteins). Once the antibody recognition has been used to isolate the nucleoprotein complex the recovered DNA can be used to make one or more probes as described herein; e.g., pull-down probes, direct monotag probes or, following restriction, indirect monotag probes.

The CHIp protocol described above may be performed using any reagent capable of binding any protein associated with a regulatory sequence or ACE, either directly or indirectly. Accordingly, binding reagents, such as antibodies, may be directed to chromatin-associated proteins, such as histones, for example, protein components of the basal transcription machinery, proteins associated with DNA replication, DNA binding proteins, such as transcription factors, and proteins present in transcriptional complexes, such as coactivators and corepressors. Specific targeted histones may include, for example, histones H1, H2A, H2B, H3, and H4. Protein components of the basal transcription machinery that may be targeted include, for example, RNA polymerases, including poll, polll and pollll, TBP and any other component of TFIID, including, for example, the TAFs (e.g. TAF250, TAF150, TAF135, TAF95, TAF80, TAF55, TAF31, TAF28, and TAF20), or any other component of the poil holoenzyme. In certain embodiments of the invention, ACEs associated with specific transcription factors, coactivators, corepressors or complexes may be isolated. Such transcription factors may include activators or repressors, and they may belong to any class or type of known or identified transcription factor. Examples of known families or structurally-related transcription factors include helix-loop-helix, leucine zipper, zinc finger, ring finger, and hormone receptors. Transcription factors may also be selected based upon their known association with a disease or the regulation of one or more genes. For example, transcription factors such as c-myc, Rel/Nf-kB, neuroD, c-fos, c-jun, and E2F may be targeted. Antibodies directed to any transcriptional coactivator or corepressor may also be used according to the invention. Examples of specific coactivators include CBP, CTIIA, and SRA, while specific examples of corepressors include the m Sin 3 proteins, MITR, and LEUNIG. Furthermore, other proteins associated with transcriptional complexes, such as the histone acetylases (HATs) and histone deacetylases (HDACs) may be targeted.

Certain illustrative strategies that may be employed in accordance with this embodiment include the following. In one example, a Chip pull-down probe can be used to query a standard array spanning some genomic sequences, for example contiguous 250 bp fragments spanning 50-100 kb of a gene locus, in order to determine the patterns of epigenetic modifications and correlate them with previously determined expression and structural data. In another example, a reiteration of the above experiment identifying ACE DNA by Chip analysis can be performed with one or more members of a comprehensive collection of antibodies having specificity for histone modifications in order to generate a detailed description of the ‘histone code’ across a locus. In another example, by preparation of the Chip-material from a range of transcriptionally permissive and non-permissive cells and tissues, or following the effects of the histone code following environmental stimuli or induction of a gene with specific chemicals, one can deduce the in vivo sequence of events which control or contribute to transcriptional regulation. In another example, the method involves assaying the effect of a class of potentially therapeutic molecules which are designed to modify the activities of the histone modifying enzymes not only on a gene of interest (as with locus profiling) but also by scanning large sections of the genome by creating in parallel an indirect monotag probe and hybridizing to appropriate tiling arrays.

In a related embodiment, multimodality profiling, e.g., combination probing with DNA modification agents, such as DNAse I, for example, and ChIP reagents, is performed using the arrays of the present invention. For example, as an alternative to performing sequential screens with DNA reagents prepared by one of the discussed selection techniques (such as sensitivity to nucleases or chemicals, selection of nucleoprotein complexes by antibodies etc.) is to perform the selections in parallel, for example performing a ChIp protocol with an antibody raised against histone H4 acetylation and then reselecting that population with a second antibody raised against a different modification. Similar combinations of Chip with nuclease/chemical sensitivity selections can be analyzed, as can the methylation status of any preselected population. ACE sequences identified and isolated from these populations can then be used in accordance with the arrays and methods described herein.

In another embodiment, alterations to the epigenetic pattern are also known to correlate with alterations with the activity of the ACEs. One of the most closely studied types of modification is cytosine methylation. The global pattern of methylation is relatively stable but certain genes become methylated if they are silenced or conversely demethylated if activated. Differential methylation can be detected by use of pairs of restriction endonucleases that cut the same site differently according to whether or not it is methylated (Tompa et al. Curr. Biol. 12: 65-68 (2002)). Alternatively it is possible to generically distinguish between a methylated and non-methylated cytosine by genomic sequencing (a methodology developed by Pfeifer et al. Science 246: 810-813 (1989)) that converts cytosine to uracil, which behaves similarly to thymine in sequencing reactions, and leaves methyl-cytosine unmodified. This material can be used as a template in PCR with primers sensitive to the C to U transition. Alternatively the potential mismatch (G:U) between oligonucleotide and template can be cleaved by E. coli Mismatch Uracil DNA Glycosylase, and that fragment removed from the population.

Additionally, in another embodiment, the enzymatic machinery which gives rise to or maintains the epigenetic patterns can also be labeled as described above so that it can be induced to cause detectable DNA modifications such as double stranded DNA breaks. Target proteins for this kind of approach would include the recently described HATs (Histone-Acetyl Transferases), HDACs (Distone De-Acetylase Complexes) whose effect on transcriptional induction has been recently described (Cell 108: 475-487 (2002)), as well as DNA methyltransferases and structural proteins that bind to the sites of methylation, such as MeCP1 and MeCP2. Histones and transcription factors are also known to become methylated, phosphorylated and ubiquinated. A range of covalent modifications, some of which have yet to be described, may be made to the structural and enzymatic machinery of transcription, replication and recombination. Current understanding indicates that such modifications have a regulatory role and it has been demonstrated that these modifications can be positively and negatively correlated with the functional activity of the underlying sequence (Science 293: 1150-1155). The potential for combinations of modifications of the ACEs overlays another layer of complexity of regulation on the underlying genome and it is possible to dynamically follow these epigenetic changes with the immunoprecipitation of the DNA sequences from in vivo nucleoprotein complexes.

ACEs define certain features of the nuclear architecture which play a large role in regulation of genomic processes. Increasingly the molecules, including proteins and RNAs, which control the structure of the nucleus are being identified, and these are also used as targets to identify ACEs.

Moreover, cytologically distinct region of interphase nuclei have been described such as the nucleoli which contain the heavily transcribed rRNA genes (Proc. Natl. Acad. Sci. USA 69: 3394-3398 (1972)) and active genes may be preferentially associated with clusters of interchromatin granules (J. Cell Biol. 131: 1635-1647 (1995)). Specific regulatory regions may become localized to distinct areas within the nucleus on transcriptional induction (Proc. Natl. Acad. Sci. USA 98: 12120-12125 (2001)). By contrast specific areas of eukaryotic nuclei have been shown to be transcriptionally inert (Nature 381: 529-531 (1996)) and associated with heterochromatin. Fractionation of the nucleus on the basis of such and similar physical properties can be used to capture sets of ACEs implicated in these processes.

ACEs

The number and location of ACEs differs between and among cell types, as may the number and identity of the proteins that bind to the genomic locale to create a given ACE. Certain ACEs may be specific to a particular tissue cell type or to a restricted set of tissue or cell types (‘Tissue-specific ACEs’). Another set may form in co-ordination with the cell cycle or due to environmental or other stimuli, including drug treatment, for example. Other ACEs may be associated with a disease or disorder. In addition, certain ACEs may be present in all tissue or cell types (‘Constitutive ACEs’) (e.g., Mol Cell Biol 1999 May;19(5):3714-26).

The total number of potential ACEs within a given cell depends largely on the cell type and state, but is generally equal to at least the number of active genes within that cell, and may be many times that number as active genes may be surrounded by or contain, e.g., their introns or other non-coding regions, more than one ACE. ACEs may function alone or in combination with other ACEs to modulate the expression of a cis-linked gene (e.g., Mol Cell Biol 1999 Nov;19(11):7600-9), or even a receptive gene in trans. Indeed, it is understood that gene regulation is generally governed by the coordinate activities of multiple regulatory elements that may be present within one or more ACEs associated with a gene locus, which includes the coding region and regulatory regions.

The superset of ACEs is expected to contain active units from virtually all known classes of genetic regulatory elements including promoters, enhancers, silencers, locus control regions, domain boundary elements, and other elements having chromatin remodeling activities. Each of the aforementioned units may in turn be comprised of one or more ACEs (e.g., Trends Genet 1999 Oct;15(10):403-8). In addition other processes may be controlled by a subset of the ACEs or interactions between them. These include, but may not be limited to, DNA replication, recombination and the structure of the genomic DNA within the nucleus such as regions of specialized chromatin structure and three-dimensional topology of the chromatin fibre. As such, the complete set of ACEs across all cells and tissue types will contain substantially all of the regulatory elements necessary to define the transcriptional program of the genome, in any state of differentiation or in response to any stimulus.

Libraries and Arrays of ACEs

A library or array of ACE sequences or sequence locations generated according to the invention provides rich and highly valuable information concerning the gene regulatory state of the cells from which the chromatin had been isolated. Further, two or more arrays or profiles (information obtained from use of an array) of such sequences are useful tools for comparing a sample set of hypersensitive sites with a reference, such as another sample, synthesized set, or stored calibrator. In using an array, individual nucleic acid members typically are immobilized at separate locations and allowed to react for binding reactions. Primers associated with assembled sets of ACEs are useful for either preparing libraries of sequences or directly detecting ACEs from other cell samples.

In many embodiments made possible from this discovery, genomic regulatory information is extracted from a biological sample without foreknowledge of genetic locus or marker information. That is, exemplified methods can identify en mass, hypersensitive sites for which no genetic marker has been identified previously. After identification, DNA containing sequences of the hypersensitive sites may be used as probes to identify complementary genomic DNA sequences to find proteins and protein complexes having regulatory activity, and to discover pharmaceutical drug activities for compounds that can influence one or multiple regulatory systems. In addition, knowledge of these sequences allow the mapping and detection of naturally occurring mutations in the genome which are implicated in causing, potentially pathogenic, changes to the transcriptional programme of the cell, such as single nucleotide polymorphisms (SNPs). In many embodiments the sequences are grouped into libraries, which can be converted or abstracted into arrays to probe multiple regulatory systems simultaneously.

A library (or array, when referring to physically separated nucleic acids corresponding to at least some sequences in a library) of ACEs has very desirable properties as further detailed below. These properties can be associated with specific cell types and cell conditions, and may be characterized as regulatory profiles. A profile, as termed here refers to a set of members that provides regulatory information of the cell from which the ACEs are obtained. A profile in many instances comprises a series of spots on an array made from deposited ACE sequences from ACEs. Without wishing to be bound by any one theory of this embodiment of the invention, it is believed that a eukaryotic cell such as a Human cell contains many potential ACEs and that only a portion of the ACE potential regulatory elements are formed at any given time. By sampling and profiling the ACEs an array presents a snapshot of the cell's regulatory status.

An array profile of a cell's regulatory status typically concerns at least 10, more preferably at least 100, 250, 500, 1000, 2000, 5,000 and even more than 10,000 ACEs in some cases. Profile information from a test sample may be more or less detailed depending on the number of ACEs required to distinguish the profile from others. For example, a profile designed to examine the presence of a particular chromosomal breakage crosslinkage or other defect may need to detect only 2-3, 2-10, 3-5, 10-20 or other small number of ACEs. With present techniques, the activation state (defined by an ability to form a nuclease hypersensitive site in chromatin) of only one or a very limited number of such sequence elements may be detected in an single experiment, such as a southern blot analysis.

In one embodiment of the invention, array profiles may be generated using random ACEs or ACEs of unknown sequence. In other embodiments, specific ACEs may be utilized, including, for example, ACEs identified as being associated with one or more genetic loci. While the sequence of ACEs used in arrays may be known, it is not necessary.

A characteristic profile generally is prepared by use of an array. An array profile may be compared with one or more other array profiles or other reference profiles. The comparative results can provide rich information pertaining to disease states, developmental state, susceptibility to drug therapy, homeostasis, and other information about the sampled cell population. This information can reveal cell type information, morphology, nutrition, cell age, genetic defects, propensity to particular malignancies and other information. Accordingly, particularly desirable embodiments were explored that use arrays for creating ACE libraries, as detailed below.

Libraries that Contain Descriptive Information of Cell Populations

The simultaneous detection of multiple hypersensitive sites using arrays provides a wide range of methods for a variety of advantages. In some embodiments, an array contains one or more internal references and the data profile is used directly without further comparison with reference data. In other embodiments, a library of sites (either sequences, position locations or both) is obtained from a sample and then compared with another library, such as a pre-existing “type” library. A type library may be characteristic for a cell type, a development status type, a disease type such as a genetic disease, or a morphologic type associated with the presence of factor(s) such as hormones, nutrients, pharmacologically active compounds and the like. The comparison to a type library may generate an output set of difference “profile information” for the library.

The term “library” as used here means a set of at least 10, preferably 50, 100, 200, 300, 500, 1000, 2000, 5000, 10,000, 20,000 30,0000 or even at least 50,000 members of nucleic acids having characteristic sequences. The library may be an information library that contains a) ACE DNA sequences, b) location information for ACEs in the genome; or c) both sequence information and matching location information. As an information library, the members preferably are stored in a computer storage medium as sequences and/or gene position locations. As a physical DNA library, the members may exist as a set of nucleic acids, clones, phages, cells or other physical manifestations of DNA in a form useful for simultaneous manipulation.

A library of nucleic acid molecules conveniently may be maintained as separate cloned vectors in host cells. Preferably each member is physically isolated from the other members, although a mixture of members within a common vessel may be suitable, particularly for assays wherein members become separated based on a physical property such as by hybridization with specific members on a solid support.

An ACE library member in most instances comprises a sequence at least 16 bases long and less than 1500 bases long. More preferably the sequence comprises between 60 bases and 400 bases. Yet more preferably the sequence comprises between 75 bases and 300 bases. The term “mean sequence length of the hypersensitive DNA sequences” means the numeric average of all DNA sequences in the respective library or array. Experimental results indicate that most ACEs are about 50 to 400 bases long and more generally about 150 to 300 bases long. However, the skilled artisan would appreciate that the length of ACEs may be quite variable, as an ACE may include one or more regulatory sequences, may be associated with different polypeptides or complexes, and/or may contain various degrees of chromatin modification. Methods for replicating DNA (or RNA) sequences and maintaining copies of those sequences in libraries are well known and have been used for some years. See for example the procedures described in U.S. Pat. Nos. 4,987,073; 5,763,239; 5,427,908; 5,853,991.

ACE Profiling and Reference Libraries

In preferred embodiments of the invention a set of at least 10 hypersensitive sequences and/or locations obtained from a sample are combined to form a profile of the sample. Typically an array is made that can detect the sequences and generate a data profile indicating at least a) the presence or absence of each sequence or ACE site in a sample or b) the relative abundance of active (hypersensitive) sites from a sample. It was discovered that “detection” of (i.e. determination of the presence and/or relative abundance of) at least some of the hypersensitive ACEs of a sample as a group profile on an array can reveal useful characteristics of the sample. Such characteristics include, for example, whether the sample contains a DNA break that increases the risk of particular malignancies or has a highly expressed region with respect to a normal state.

In another embodiment, a sample is processed to determine ACE usage and a profile is obtained from binding reactions between nucleic acid sequences obtained from the sample and other nucleic acid references. Advantageously either the reference nucleic acids or the sample nucleic acids are first bound in an array and the array exposed to the other set. In an embodiment at least 10, more preferably at least 100, 1000, 10,000, or even more than 20,000 reference nucleic acids are used in this embodiment.

In yet another embodiment a sample is processed to generate nucleic acids corresponding to sequences of ACEs and the nucleic acids identified by sequencing, mass spectrometry and/or another method. Profile results obtained advantageously are compared to known values.

Yet another embodiment of the invention provides a master organism reference library that contains a large collection, e.g., greater than 100, greater than 10,000 or greater than 25,000 ACE sequences representative of the organism. In one embodiment, the library substantially contains all possible assayable ACEs of a cell. The phrase “substantially contains” in this context means at least 10% and preferably at least 50% of all possible hypersensitive sites, including every site that can be found in one situation (cell type, cell morphology, or other condition) or another. Preferably “substantially contains” refers to at least 75% of all possible hypersensitive sites, and more preferably refers to at least 90%, 95% and even at least 99% of all sequences and/or site locations. In an embodiment such library is made by mapping ACEs from at least 3 different cell types of an organism and more preferably 4, 5, 6, or even more than 10 types of different cells, and compiling all of the different ACEs into a “organism specific” set of ACEs. One version of a library includes sequences corresponding to each ACE. Yet another version of the library includes position information of each ACE. Either or both versions of data are very useful tools for diagnostic tests and other studies.

Yet another embodiment is a cell type specific reference library that “substantially contains” all ACEs of that specific type of cell. Another related embodiment is a library prepared from a cell or cells treated with an external stimuli, such as a drug or environmental stimuli, for example. External stimuli may include any compound, such as drugs, small molecules, hormones, cytokines, etc., and any other types of treatment or stimulation, such as changes in environmental factors, e.g. temperature, pressure, or atmosphere, and including radiation, for example. The term “substantially contains” in this context means at least 10% and preferably at least 50% of all ACEs that behave as hypersensitive sites under one or more conditions experienced by that cell type. More preferably, “substantially contains” refers to at least 75% of all possible hypersensitive sites, and even more preferably refers to at least 90%, 95% and even at least 99% of all sequences and/or site locations. By way of example, a human cell line was found to contain approximately 30,000 hypersensitive site ACEs, when examined in late log stage of growth.

In certain embodiments, libraries and arrays of the invention may contain ACEs associated with one or more specific genes or genetic loci, including, e.g. genes known to be associated with diseases or other disorders.The invention further includes novel methods of tagging or labeling polynucleotides, which are applicable for a variety for purposes, including, e.g. probing arrays of the invention. These methods of tagging or labeling polynucleotides are described in further detail below, and include the preparation of (1) fixed length direct monotags, (2) fixed length indirect monotags, (3) direct pull down probes, and (4) labeled chromatin probes. The skilled artisan would understand that the exemplary methods described in general below and more specifically in the accompanying Examples may be modified in certain respects, according to principles and techniques known in the art, to achieve essentially the same results, and the invention encompasses all such modifications and variations of the described procedures.

(1) Fixed Length Direct Monotags

Direct monotags map precisely to either strand of a breakage in the DNA. The breakpoints are typically captured by the ligation of either a blunt or T-tailed linker following repair of the breakage site and Taq-polymerase mediated A-tailing. The linker brings a cutting site for a type IIs restriction endonuclease so it is adjacent to the breakage site. Type IIs restriction endonucleases have the property of cutting a site distal from their recognition site, an example of which is MmeI which cuts 20 nt and 18 nt on the top and bottom strands respectively away from its binding site. This action creates a ‘monotag,’ a snippet of genomic sequence associated with a particular event in the genome, for example, a DNA breakage caused by the introduction of exogenous nucleases. The sequence is of sufficient length to in general allow the majority of them to be mapped uniquely to the genome, or in the context of arrays hybridize specifically to a target sequence.

Some cutting agents will produce breakages with specific features that can be specifically targeted by the linker. Examples of these would include: cutting with DNaseI in the presence of manganese as the divalent cation to produce a predominance of blunt ends; treating nuclei with a restriction enzyme to digest the subpopulation of restriction sites that are accessible in the chromatin (essentially those with fortuitous placements in ACEs) to generate a ‘sticky end’ to which a linker can be ligated. One specific advantage of these approaches is that they do not label breakages which are introduced in a quasi-random fashion in the process of extracting the genomic DNA from the nuclei, this is a considerable source of experimental background.

As the monotags can be derived from strands on either side of the breakage, the system contains an internal control to help screen false positive results. That is, if the probe successfully identifies one target on the array with a certain efficiency, it will be predicted to detect a second target corresponding to the sequence from the other side of the breakage with a similar efficiency.

When that breakage is created by the action of a footprinting reagent, such as DNaseI, hyrdoxyradical reagents or the like, the distribution of monotags can be used to recreate a ‘footprint’ on a specially designed tiling array. The tiling array is so designed that every target polynucleotide, typically each the same size, corresponds to a specific region of DNA, with different targets containing DNA sequences corresponding to shifts of one or more nucleotides relative to each other. For example, a tiling array may be designed such that a target of a 35 nucleotide (or window of some size) stretch of genomic sequence differs from its adjacent target by a shift of a single base pair, so that a series of targets will represent a moving window across the genomic region. If mapping of a lower resolution is required, for example, by using micrococcal nuclease, the digestion pattern of which gives information about the distribution of entire nucleosomes in the chromatin, potentially the gap between the position of the adjacent sequences can be increased; so they are shifted by 5 bp each, or are adjacent but share no overlap, or even are not contiguous sequences. Thus, the invention contemplates overlapping targets with as little as one nucleotide shifts and as large as the entire size of the target, as well as non-overlapping targets. Overlaps may also be of any intermediate size, such as 5 nucleotides, 10 nucleotides, 20 nucleotides, 30 nucleotides, 50 nucleotides, 100 nucleotides, 200 nucleotides, or any intermediate integer value between.

(2) Fixed Length Indirect Monotags

Indirect monotags typically map the closest chosen restriction site to the DNA breakage. An example of this procedure is that the breakage site is captured either by direct enzymatic biotinylation, with terminal transferase and biotin-ddUTP, or by ligation of a linker. Following this step, the genomic DNA is cut with a restriction enzyme, NlaIII for example, and a second linker is ligated to that site. It is this linker which contains the restriction site for a type IIs restriction enzme and cleavage with this creates a population of Indirect monotags.

The advantage of this approach is that it allows the experimenter to control the resolution of the experiment and hence the number of data points that need to be collected. While sampling a large space like the human genome with Direct monotags represents 3×10⁹ potential cut sites (to give 1 bp resolution), choosing to map to the nearest 4-cutter restriction enzyme, such as NlaIII, reduces the sample size to approximately 12 million (the predicted number of NlaII sites) with an average resolution of 250 bp. As for the Direct monotags, the probe population is internally controlled, and the efficiency of detecting NlaIII sites either side of a breakage should be similar. In certain embodiments, Tiling microarrays may be constructed where a 100 kb stretch can be profiled with an estimated 400 oligonucleotide sequences (typically these can be manufactured with 60 nt stretches which correspond to the 25 nucleotides either side of an NlaIII site). Such arrays would allow either de novo discovery of ACEs within that genomic stretch, or, if the sequences are bio-informatically extracted from sequences we have cloned, then the tiling arrays could be used as a validation step for libraries of the invention.

Mapping to the closest NlaIII sites is an efficient way of searching for or validating ACES that are of a similar size. Another application of this embodiment of the invention is the study of larger features within the genome, such as deletions of large genomic (e.g. greater than 0.1 Mbp) within clinical populations. In this scenario, the genomic DNAs are digested with a rare restriction cutter, such as Sse8387I (which produces fragments with an average size of 30 kbp), and the linkers are ligated directly to that site. Cutting from the MmeI site within that linker creates a monotag that can be used to screen and used to make the monotags.

(3) Direct Pull Down Probes

In this version of preparing probes, the breakage site is again either enzymatically labeled (as described above) or ligated to a biotinylated linker. Following a purification step to remove unincorporated biotin substrates, the genomic DNA is cut with a restriction enzyme. The majority of the genome will be contained within the simple restriction fragments and as they have not been labeled with biotin will not be captured on a separation system, such as paramagnetic beads coated with strepavidin. The biotinylated ends, marking the breakage sites, are captured, and this fraction is then taken forward to be labeled in order to create a probe population.

Modifications can be made to the process whereby in place of the restriction digest of the genomic DNA it is randomly broken, either by physical shearing, sonication or treatment with non-specific or low-specificity cutters of naked DNA, such as DNaseI. These protocols have advantage that they are rapid and reproducible.

(4) Probes Made from Labeling of Chromatin Fractions

Sucrose gradient centrifugation or other preparative methods can be used to isolate discrete fractions of treated genomic DNAs according to their mass. These fractions can then be labeled directly to produce probes or used as a source for monotag populations. The rationale for this approach is that it is more likely that smaller fragments will contain a genuine cutting site for an ACE than not, i.e. it consists of two random background cuts. Certainly, the ability to remove the vast majority of high molecular weight DNA considerably reduces the background due to isolated random breakages (either caused by the action of the exogenously added enzyme or shearing due to handling).

A variety of different targets and probes have been described and may be used according to the invention, in any combination. In certain embodiments, targets and/or probes may be of a fixed length, while in other embodiments targets and/or probes may be of variable length. Accordingly, in specific embodiments, combinations of the invention include fixed target and fixed probe lengths, variable target and fixed probe lengths, fixed target and variable probe lengths, and variable target and variable probe lengths.

Generation and Use of Library Members in MicroArrays

Many uses of the invention arise from the ability to generate, manipulate and analyze large amounts of information through libraries and their use in microarrays to provide information. Arrays generally are made and used by a variety of methods that can be discussed in terms of i) preparation of arrays; ii) sample preparation and conversion into fragment libraries, iii) manipulating the fragments by, for example, amplifying and cloning them, and iv) profiling libraries (i.e. either the entire set of prepared fragments or a subset of them) by detection on arrays.

i. Preparation of Arrays Containing ACEs

Microarrays, also called “biochips” or “arrays” are miniaturized devices typically with dimensions in the micrometer to millimeter range for performing chemical and biochemical reactions and are particularly suited for embodiments of the invention. Arrays may be constructed via microelectronic and/or microfabrication using essentially any and all techniques known and available in the semiconductor industry and/or in the biochemistry industry, provided only that such techniques are amenable to and compatible with the deposition and screening of polynucleotide sequences.

Microarrays are particularly desirable for their virtues of high sample throughput and low cost for generating profiles and other data. A DNA microassay typically is constructed with spots that comprise nucleic acid with ACE sequences. In a preferred embodiment immobilized DNAs have sequences that hybridize to ACE hypersensitive sites such as putative genomic regulatory elements.

Microarrays according to embodiments of the invention may include immobilized biomolecules such as oligonucleotides, cDNA, DNA binding proteins, RNA and/or antibodies on their surfaces. Advantageous embodiments of the invention have immobilized nucleic acid on their surfaces. The nucleic acid participates in hybridization binding to nucleic acid prepared from hypersensitive sites. Such chips can be made by a number of different methodologies. For example, the light-directed chemical synthesis process developed by Affymetrix (see, U.S. Pat. Nos. 5,445,934 and 5,856,174) may be used to synthesize biomolecules on chip surfaces by combining solid-phase photochemical synthesis with photolithographic fabrication techniques. The chemical deposition approach developed by Incyte Pharmaceutical uses pre-synthesized cDNA probes for directed deposition onto chip surfaces (see, e.g., U.S. Pat. No. 5,874,554).

Other useful technology that may be employed is the contact-print method developed by Stanford University, which uses high-speed, high-precision robot-arms to move and control a liquid-dispensing head for directed cDNA deposition and printing onto chip surfaces (see, Schena, M. et al. Science 270:467-70 (1995)). The University of Washington at Seattle has developed a single-nucleotide probe synthesis method using four piezoelectric deposition heads, which are loaded separately with four types of nucleotide molecules to achieve required deposition of nucleotides and simultaneous synthesis on chip surfaces (see, Blanchard, A. P. et al. Biosensors & Bioelectronics 11:687-90 (1996)). Hyseq, Inc. has developed passive membrane devices for sequencing genomes (see, U.S. Pat. No. 5,202,231). These methods and adaptations of them as well as others known by skilled artisans may be used for embodiments of the invention.

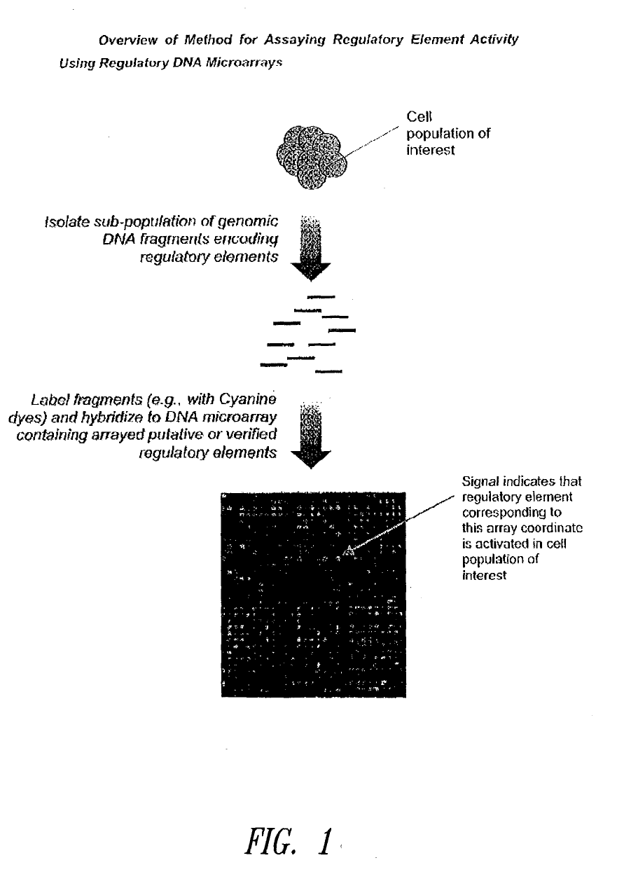

Arrays generally may be of two basic types, passive and active. Passive arrays utilize passive diffusion of sample molecule for chemical or biochemical reactions. Active arrays actively move or concentrate reagents by externally applied force(s). Reactions that take place in active arrays are dependant not only on simple diffusion but also on applied forces. Most available array types, e.g., oligonucleotide-based DNA chips from Affymterix and cDNA-based arrays from Incyte Pharmaceuticals, are passive. Structural similarities exist between active and passive arrays., Both array types may employ groups of different immobilized ligands or ligand molecules. The phrase “ligands or ligand molecules” refers to bio/chemical molecules with which other molecules can react. For instance, a ligand may be a single strand of DNA to which a complementary nucleic acid strand hybridizes. A ligand may be an antibody molecule to which the corresponding antigen (epitope) can bind. A ligand also may include a particle with a surface having a plurality of molecules to which other molecules may react. Preferably the reaction between ligand(s) and other molecules is monitored and quantified with one or more markers or indicator molecules such as fluorescent dyes. In preferred embodiments a matrix of ligands immobilized on the array enables the reaction and monitoring of multiple analyte molecules. For example, an array having an immobilized library of ACE fragments may be tested for binding with one or more putative DNA binding proteins. A two dimensional array is particularly useful for generating a convenient profile that may be imaged, as exemplified in FIGS. 1 through 6.

More recent developments in array manufacture and use are specifically contemplated. For example, electronic arrays developed by Nanogen can manipulate and control sample biomolecules by electrical fields generated with microelectrodes, leading to significant improvement in reaction speed and detection sensitivity over passive arrays (see, U.S. Pat. Nos. 5,605,662, 5,632,957, and 5,849,486). Another active array procedure contemplated in some embodiments is the technology described in U.S. Patent No. 6,355,491 and issued to Zhou et al. entitled “Individually addressable micro-electromagnetic unit array chips.” This latter technology provides an active array wherein individually addressable (controllable) units arranged in an array generate magnetic fields. The magnetic forces manipulate magnetically modified molecules and particles and promote molecular interactions and/or reactions on the surface of the chip. After binding, the cell-magnetic particle complexes from the cell mixture are selectively removed using a magnet. (See, for example, Miltenyi, S. et al. “High gradient magnetic cell-separation with MACS.” Cytometry 11:231-236 (1990)). Magnetic manipulation also is used to separate tagged ACE sequences during sample preparation in desirable embodiments, before application of DNA to a test array.

Arrays can be used to compare reference libraries as well as profiling based on as little as a single nucleotide difference. The chemistry and apparatus for carrying out such array profiling and comparisons are known. See for example the articles “Rapid determination of single base mismatch mutations in DNA hybrids by direct electric field control” by Sosnowski, R. G. et al. (Proc. Natl. Acad. Sci., USA, 94:1119-1123 (1997)) and “Large-scale identification, mapping and genotyping of single-nucleotide polymorphisms in the Human genome” by Wang, D. G. et al. (Science, 280:1077-1082 (1998)), which show recent techniques in using arrays for manipulation and detection of sequence alternations of DNA such as point mutations. “Accurate sequencing by hybridization for DNA diagnostics and individual genomics.” by Drmanac, S. et al. (Nature Biotechnol. 16:54-58 (1998)), “Quantitative phenotypic analysis of yeast deletion mutants using a highly parallel molecular bar-coding strategy” by Shoemaker, D. D. et al. (Nature Genet., 14:450-456 (1996)), and “Accessing genetic information with high density DNA arrays.” by Chee, M et al., (Science, 274:610-614 (1996)) also show known array technology used for DNA sequencing.

Further examples of technology contemplated for use in making and using arrays are provided in “Genome-wide expression monitoring in Saccharomyces cerevisiae.” by Wodicka, L. et al. (Nature Biotechnol. 15:1359-1367 (1997)), “Genomics and Human disease—variations on variation.” by Brown, P. O. and Hartwell, L. and “Towards Arabidopsis genome analysis: monitoring expression profiles of 1400 genes using cDNA microarrays.” by Ruan, Y. et al. (The Plant Journal 15:821-833 (1998)).Additional microarray technologies that may be utilized according to the present invention include, for example, electronic microarrays, including, e.g. the NanoChip Electronic Microarray, which is available from Nanogen, Inc. (San Diego, Calif.) and described in detail in U.S. Pat. No. 6,258,606, “Multiplexed Active Biologic Array”; U.S. Pat. No. 6,287,517, “Laminated Assembly for Active Bioelectronic Devices”; U.S. Pat. No. 6,284,117, “Apparatus and Method for Removing Small Molecules and Ions from Low Volume Biological Samples”; U.S. Pat. No. 6,280,590, “Channel-Less Separation of Bioparticles on a Bioelectronic Chip by Dielectrophoresis”; and U.S. Pat. No. 6,254,827, “Methods for Fabricating Multi-Component Devices for Molecular Biological Analysis and Diagnostics, and references cited therein, all of which are incorporated by reference in their entirety.

Methods of the invention may further include nanopore technologies developed by Harvard University and Agilent Technologies, including, e.g. nanopore analysis of nucleic acids. Nanopore technology can distinguish between a variety of different molecules in a complex mixture, and nanopores can be used according to the invention to readily sequence nucleic acids and/or discriminate between hybridized or unhybridized unknown RNA and DNA molecules, including those that differ by a single nucleotide only. Nanopore technology is described in U.S. Pat. No. 6,015,714, “Characterization of individual polymer molecules based on monomer-interface interactions,” related patents and applications, and references cited within, all of which are incorporated by reference in their entirety.

In certain embodiments, the invention may employ surface plasmon resonance technologies, such as, for example, those available from Biocore International AB, including the Biacore S51 instrument, which provides high quality, quantitative data on binding kinetics, affinity, concentration and specificity of the interaction between a compound and target molecule. Surface plasmon resonance technology provides non-label, real-time analysis of biomolecular interactions and may be used in a variety of aspects of the present invention, including high throughput analysis of microarrays. Surface plasmon resonance methods are known in the art and described, for example, in U.S. Pat. No. 5,955,729, “Surface plasmon resonance-mass spectrometry” and U.S. Pat. No. 5,641,640, “Method of assaying for an analyte using surface plasmon resonance,” which also describes analysis in a fluid sample, which are incorporated by reference in their entirety.

Microarrays of the invention include, in certain embodiments, peptide nucleic acid (PNA) biosensor chips. PNA is a synthesized DNA analog in which both the phosphate and the deoxyribose of the DNA backbone are replaced by polyamides. These DNA analogs retain the ability to hybridize with complementary DNA sequences. Because the backbone of DNA contains phosphates, of which PNA is free, an analytical technique that identifies the presence of the phosphates in a molecular surface layer would allow the use of genomic DNA for hybridization on a biosensor chip rather than the use of DNA fragments labeled with radioisotopes, stable isotopes or fluorescent substances. A major advantage of PNA over DNA is the neutral backbone and the increased strength of PNA/DNA pairing. The lack of charge repulsion improves the hybridization properties in DNA/PNA duplexes compared to DNA/DNA duplexes, and the increased binding strength usually leads to a higher sequence discrimination for PNA-DNA hybrids than for DNA-DNA.

Arrays of the invention may be prepared by any available means and may contain a variety of different samples, e.g. polynucleotide sequences. In certain embodiments, these polynucleotide sequences may correspond to some or all of all ACEs within a cell. In other embodiment, particular ACEs or genomic sequences may be selected. In one embodiment, sequences of specific genes may be used, such as, for example, sequences associated with a particular cell type, disease state, environmental or other stimuli (e.g. chemical), or developmental stage. In addition, sequences corresponding to a particular region of genomic DNA, such as a gene locus, may be used on an array. Such sequences may cover all or substantially all of a gene locus, and may include coding sequences as well as regulatory and other non-coding sequences.

In certain embodiments, arrays may comprise reduced information sets as compared to arrays comprising substantially all ACEs associated with a cell. Such reduced information sets may be selected based on sequence or genomic location, as described supra, or they may be selected by other means. For example, reduced information set arrays may comprise sequences isolated using particular restriction enzymes and, therefore, may comprise, in specific examples, only 4-cutter-proximal regions or regions proximal to rare cutter restriction sites, which may span large regions.

In one embodiment, repetitive sequences are removed from the arrayed polynucleotides or probes. Repetitive sequences may be removed prior to deposition on an array platform by any means available in the art. For example, repetitive sequences may be adsorbed from a mixture, as described, for example, in Grandori, C. et al,. EMBO J 15:4344-57 1996). In another embodiment, repetitive sequences, e.g. genome-specific repetitive sequences may be removed using an algorithm (need reference). In another embodiment, repetitive sequences may be identified and arrayed. The identification of repetitive sequences then allows them to be removed from profiled produced from the arrays, if desired.

Generally, repeitive sequences may be removed at three levels:

1) Bio-informatically: Algorithms and public engines such as Repeatmasker may be used to identify target sequences which have a high repetitive content. RepeatMasker is a program that screens DNA sequences for interspersed repeats known to exist in mammalian genomes as well as for low complexity DNA sequences. The output of the program is a detailed annotation of the repeats that are present in the query sequence as well as a modified version of the query sequence in which all the annotated repeats have been masked (replaced by Ns). On average, over 40% of a human genomic DNA sequence is masked by the program. Sequence comparisons in RepeatMasker are performed by the program cross_match, an implementation of the Smith-Waterman-Gotoh algorithm (Smit, AFA & Green, P RepeatMasker at http://ftp.genome.washington.edu/RM/RepeatMasker.html). Optionally, identified sequences may be not placed on the arrays.

2) Repetitive sequences may be removed in the hybridization reaction by inclusion of a competitor agent such as Cot1.

3) Repetitive sequences may be removed in the preparation of the probe by doing a subtraction step. For example, Cot1 DNA, or versions of human repetitive elements created by performing PCR with biotinylated degenerate oligos designed to amplify this class of molecules, could be treated with a reagent such as photobiotin, for examepl, then an excess of this could be hybridized with a non-biotinylated probe population, followed by extraction of all of the biotinylated DNA on Dynal beads. The flowthrough would represent repetitive-depleted probe.

Array hybridisations using probes from which repetitive DNA weas removed will light up the repetitive control spots on the arrays less intensively than a probe simply made from genomic DNA. Furthermore, targetting the DNaseI cut sites should be sufficient to ensure a depletion in repetitive elements.

ii. Interrogation of Arrays Containing ACEs (Sample Preparation via Marking ACEs and Conversion into Fragment Libraries).

A first step in the generation and use of library members is to mark multiple hypersensitive sites. A site may be marked, for example, by a biochemical alteration that can be used to identify or separate the site for sequencing. This alteration often will involve breaking or making a covalent bond within specific ACEs. For example, a nuclease may mark by cutting the ACE. In a preferred embodiment non-specific nuclease such as DNAse I cuts DNA at the hypersensitive sites.

In a particularly desirable embodiment DNAse I is used to mark hypersensitive sites by cutting DNA strands at these sites. Following isolation and optional amplification of the DNA segments that flank the hypersensitive cut sites, the fragments are sub-cloned into a suitable vector, such as a commercially available bacterial plasmid. To effect this, the fragments are digested with restriction enzymes, cut sites of which have been engineered into the linker regions. Following incorporation into suitable bacterial plasmids, colonies are recovered which contain bacteria in which the plasmid replicates.

Other agents and methods that may be used to mark eukaryotic DNAs at hypersensitive sites include, for example, radiation such as ultraviolet radiation, chemical agents such as chemotherapeutic compounds that covalently bind to DNA or become bound after irradiation with ultraviolet radiation, other clastogens such as methyl methane sulphonate, ethyl methone sulphonate, ethyl nitrosourea, Mitomycin C, and Bleomycin, enzymes such as specific endonucleases, non-specific endonucleases, topoisomerases, such astopoisomerase II, single-stranded DNA-specific nucleases such as S1 or P1 nuclease, restriction endonucleases such asEcoR1, Sau3a, DNase 1 or Styl, methylases, histone acetylases, histone deacetylases, and any combination thereof.

As will be appreciated by skilled artisans, clastogens may be used to break DNA and the broken ends tagged and separated by a variety of techniques. Compounds that covalently attach to DNA are particularly useful as conjugated forms to other moieties that are easily removable from solution via binding reactions such as biotin with avidin. The field of antibody or antibody fragment technology has advanced such that antibody antigen binding reactions may form the basis of removing labeled, nicked or cut DNA from a hypersensitive ACE site.

In many embodiments, after forming a break or directly binding to the DNA, the affected DNA sequence around the site may be isolated and determined and/or the site mapped to a location in the genome. For example, an agent that forms a covalent bond with DNA may be conjugated to a binding member such as biotin or a hapten. After bond formation, endonuclease may be used to generate smaller DNA fragments. Fragments that contain the marked ACE may be isolated by a specific binding reaction with a conjugate binding member (avidin or an antibody/antibody fragment respectively in this case), for example, on a solid phase that immobilizes the ACE fragments and allows removal of the other fragments.

Sample preparation begins with chromatin from cellular material. Preferably the chromatin is extracted from a eukaryotic cell population such as a population of animal cells, plant cells, virus-infected cells, immortalized cell lines, cultured primary tissues such as mouse or Human fibroblasts, stem cells, embryonic cells, diseased cells such as cancerous cells, transformed or untransformed cells, fresh primary tissues such as mouse fetal liver, or extracts or combinations thereof. Chromatin may also be obtained from natural or recombinant artificial chromosomes. For example, the chromatin may have been assembled in vitro using previously sub-cloned large genomic fragments or Human or yeast artificial chromosomes.

In many embodiments multiple ACE sequences and/or location sites are obtained from a eukaryotic cell sample by first extracting and purifying nuclei from the sample as for example, described in U.S. Pat. No. 09/432,576. Briefly, a sample is treated to yield preferably between about 1,000,000 to 1,000,000,000 separated cells. The cells are washed and nuclei removed, by for example NP-40 detergent treatment followed by pelleting of nuclei. An agent that preferentially reacts with genomic DNA at ACEs is added and marks the DNA, typically by cutting or binding to the DNA. In a particularly advantageous embodiment DNAse I is used to form two single strand breaks near each other, and typically within 5 bases of each other. After reaction with hypersensitive DNA sites the reacted DNA is, if not already, converted into smaller fragments and the reacted fragments optionally are amplified and separated into a library. Preferably breaks on both strands within up to 10 base pairs from each other are detected after extraction by cloning one or both sides of the site.

iii. Manipulation of Fragments

Isolation of DNA after marking and fragmentation may be accomplished by a number of techniques. Exemplary methods include: adaptive cloning linkers that facilitate selective incorporation into a cloning vector or PCR; streptavidin/biotin recovery systems; magnetic beads, silicated beads or gels; dioxygenin/anti-dioxygenin recovery systems; or a variety of other methods. Once isolated (or even before isolation), fragments can be labeled with a detectable label. Suitable detectable labels include fluorescent chemicals, magnetic particles, radioactive materials, and combinations thereof.

Amplification of isolated DNA fragments may be required in the event that the quantities of DNA recovered from this isolation step are insufficient to effect efficient cloning of the desired segments, or simply to produce a more efficient process.

In a desirable embodiment described in Example 1 a biotin-labeled linker is added after formation of cut ends by DNase I and binds to the cut ends. The mixture is digested with one or more restriction endonucleases such as Sau3a or Styl to create smaller fragments and the biotin labeled fragments recovered by a binding reaction to immobilized avidin followed by removal of unbound fragments. An amplification step such as polymerase chain reaction (“PCR”) optionally may be performed. To render the fragments fit for PCR, another linker can be incorporated at the opposite end from that of the biotinylated linker.

Newer variations of PCR and related DNA manipulations such as those described in U.S. Pat. Nos. 6,143,497 (Method of synthesizing diverse collections of oligomers); U.S. Pat. No. 6,117,679 (Methods for generating polynucleotides having desired characteristics by iterative selection and recombination); U.S. Pat. No. 6,100,030 (Use of selective DNA fragment amplification products for hybridization based genetic fingerprinting, marker assisted selection, and high throughput screening); U.S. Pat. No. 5,945,313 (Process for controlling contamination of nucleic acid amplification reactions); U.S. Pat. No. 5,853,989 (Method of characterization of genomic DNA); U.S. Pat. No. 5,770,358 (Tagged synthetic oligomer libraries); U.S. Pat. No. 5,503,721 (Method for photoactivation); and U.S. Pat. No. 5,221,608 (Methods for rendering amplified nucleic acid subsequently un-amplifiable) are desirable. The contents of each cited patent which pertains to methods of DNA manipulation are most particularly incorporated by reference.

DNA samples thus prepared by marking and amplification may be further manipulated and applied to an array in a number of ways. For example, the DNA sequence may be amplified using the polymerase chain reaction from a library containing such sequences, and subsequently deposited using a microarraying apparatus. In another way the DNA sequence is synthesized ex situ using an oligonucleotide synthesis device, and subsequently deposited using a microarraying apparatus. In yet another way the DNA sequence may be synthesized in situ on the microarray using a method such as piezoelectric deposition of nucleotides. The number of sequences deposited on the array generally may vary upwards from a minimum of at least 10, 100, 1000, or 10,000 to between 10,000 and several million depending on the technology employed.

A DNA fragment subpopulation containing ACE sequences advantageously may be detected by fluorescence measurements by labeling with a fluorescent dye or other marker sufficient for detection through an automated DNA microarray reader. The labeled fragment population generally is incubated with the surface of the DNA microarray onto which has been spotted different binding moieties and the signal intensity at each array coordinate is recorded. Fluorescent dyes such as Cy3 and Cy5 are particularly useful for detection, as for example, reviewed by Integrated DNA Technologies (see “Technical Bulletin at http://www.idtdna.com/ program/tech bulletins/Dark_Quenchers.asp) and as provided by Amersham (See Catalog#PA53022, PA55022 and related description).

DNA arrays that contain sequences such as those described here, their complementary sequences, or other sequences derived from them may be prepared, analyzed and/or profiledby any of a wide variety of approaches and technologies, certain illustrative examples of which are provided hereinbelow.

B. Methods of ACE Profiling

As described above libraries may exist in silico as DNA sequences or in vitro as physical elements that contain DNA. In other embodiments libraries are profiled on arrays. Data obtained from large assemblages of library elements are useful for many purposes. In principle, two or more arrays are prepared under similar conditions with one array acting as a control or reference for the other(s). For example, alteration of expression induced by a test compound such as a drug candidate may be determined by creating two arrays, one that corresponds to cells that have been treated with the test compound and a second that corresponds to the cells before treatment.

Differences in array data profiles can reveal which ACEs are affected by the test compound. An ACE may be more hypersensitive in the presence of the drug, as seen by more abundant hits at that ACE site during the nuclei incubation/reaction step leading to a stronger ACE signal in a profile. An ACE may be found less hypersensitive if, in comparison to a no drug control, a weaker signal was produced for that ACE spot in the array. In another example, an array profile obtained from a malignant tissue sample may be compared with an array profile obtained from a control or normal tissue sample. An inspection of the hypersensitive ACE differences between the arrays may reveal a genetic cause in the disease or a genetic factor in the disease progression.

An ACE profile may be as simple as a small set of 6, 7, 8, 10,10 to 25, 25 to 100, or 100 to 500 ACEs. The procedures and materials illustrated in “Cystic fibrosis mutation detection by hybridization to light-generated DNA probe arrays.” by Cronin, M. T. et al. (Human Mutation, 7:244-255 (1996)), and “Polypyrrole DNA chip on a silicon device: Example of hepatitis C virus genotyping.” by Livache, T. et al. (Anal. Biochem. 255:188-194 (1998)) are particularly contemplated for determining differences between a reference sequence or library sequence and that obtained from a sample. These documents are specifically incorporated by reference and illustrate the knowledge of skilled artisans in this field.

In another embodiment an array generates data that reveal ACE copy number. As will be readily appreciated, some ACEs are more hypersensitive than others for a given cell state and this character can be seen as a higher copy number, or (where appropriate) a greater detection signal compared to another ACE or reference sample. According to an embodiment of the invention, the relative copy numbers of one or more ACEs are compared to a reference or set of references to determine a relative activity of the ACE.

Without wishing to be bound by any one theory of this embodiment of the invention, it is believed that ACE profiling in this manner often yields a more accurate determination of gene regulation than measuring transcribed mRNA or a protein product of a gene because “hypersensitivity” itself is a more direct measure of whether a regulatory system is on or off. In contrast, mere quantitation of a transcription or translation product generally reflects more variables and may be less tightly associated with the biochemical operation of the corresponding regulatory unit. One embodiment of the invention is an improvement in previous diagnostic and quantitative tests for gene regulation wherein one or more ACEs and/or a ACE profile is determined by an array and correlated with a particular protein function or other biological effect.

Another embodiment of the invention is a set of primers corresponding to a library of ACEs and which can form an array. Preferably the library contains at least 10, 100, 250, 500, 1,000, 5,000 or even more than 10,000 primers that correspond to specific ACEs. In an advantageous method a library of ACE specific primers are used to selectively amplify or detect ACE sequences corresponding to a particular desired profile. A library profile may be as small as a set of 5 or 10 ACE sequences. In this case 5 or 10 primers with sequences corresponding to the desired ACEs may be used with a DNA sample to selectively amplify those ACEs for further analysis.

The library profiling and comparison techniques of the invention are useful for discovery of drugs that interact with regulatory mechanisms mediated by one or more ACEs. A respective embodiment directly screens for drugs by exposing a microarray of ACE sequences to potential drugs. Another embodiment scores the effect of a chemical on an intact nucleus by exposing the nucleus to the drug and then deriving a library of ACEs from the treated nucleus. Representative techniques and materials useful in combination for this embodiment are found in “Selecting effective antisense reagents on combinatorial oligonucleotide arrays.” by Milner, N. et al. (Nature Biotechnol., 15:537-541 (1997)), and “Drug target validation and identification of secondary drug target effects using DNA microarray.” by Marton, M. J. et al. (Nature Medicine, 4:1293-1301 (1998)).

While many embodiments of the invention concern profiled information from arrays, the fragment libraries and derivatives of them are independently valuable tools. A fragment library prepared by marking and separating out ACEs from chromatin contains valuable information that may be extracted and used in a variety of forms. For example, the fragments can be sequenced and their profile information entered into a computer or other data base for comparison in silico with one or more reference libraries. In addition, an ACE fragment can be used to identify and isolate one or more coding regions with which the ACE sequence is associated. Moreover, the fragments may be cloned and used for drug discovery via one or more screening techniques described herein and apparent to an artisan of ordinary skill in view of the instant disclosure. Isolated fragments may be cloned by any of a number of techniques using any number of cloning vectors. Exemplary techniques include: introduction into self-replicating bacterial plasmid vectors; introduction into self-replicating bacterophage vectors; and introduction into yeast shuttle vectors.

Generally, the fragment library may be converted by an array manipulation in silico or in vitro into other valuable libraries by a variety of techniques. For example, members of the library having highly repetitive sequences may be deleted from computer memory by pattern matching and removal of matched sequences. Highly repetitive sequences and/or other undesirable sequences/sites such as those found by random breaks during DNA isolation. Such fragment libraries, either as computer data base set or as physical DNA containing sets of vessels, molecules, plasmids, cells or organisms, are valuable items of commerce. For example, a library obtained from tissue of a patient with a particular disease will represent a snapshot of the active ACE profile associated with the disease and has significant value for drug discovery and for diagnosis. Both a computer based data set library and physical embodiments of that set such as a library of clones has great utility and may be sold for a variety of purposes.

In view of the various array-based library screening methods described herein, it will be appreciated by the artisan of skill in the art that the disclosed methods for generating ACE profiles, and the ACE profiles so obtained, provide valuable sources of novel and important biological information. Indeed, a number of important advantages of the present invention stem from the ability to readily compare ACE profiles in biological samples., e.g., at different developmental stages, across different cell types, in different disease states, and/or in response to candidate therapeutic compounds, etc.

For example, in one embodiment, the present invention provides a method for profiling cell or tissue samples. ACE profiles are first generated from one or more test samples and the profiles so obtained are then compared to a reference profile in order to identify differences in ACE activity between the two samples. The identification of one or a plurality of ACEs that is characteristic of a given disease state relative to a healthy control state, for example, provides important diagnostic information about the disease state. In one example, ACE profiles are generated in accordance with the present invention for at least two samples or sets of samples, one representing healthy control tissue and the other representing diseased human tissues, in order to identify ACE activity that is altered in the disease state. The invention thus provides methods for identifying ACE profiles that are associated with, and thereby diagnostic for, a disease state, such as cancer. For example, ACE profiles can be generated for a collection of samples, e.g., breast cancer samples, and compared to a suitable reference profile such as a profile generated from normal healthy tissue of the same type from which the cancer sample was derived, i.e., normal breast tissue. Alterations in activity of an individual ACE sequence, or in a pattern of ACE activities, can be readily detected and quantitated by the array profiling methods described herein to identify a “signature” profile of ACE activity that is characteristic of, and preferably diagnostic for, the disease. The activity of individual ACEs and/or the activity of a group or pattern of ACEs, is thus correlated with the occurrence of the particular disease state. In this way, tissue profiling identifies ACE sequences and groups of sequences that have utility in methods for the diagnosis and/or monitoring of the disease state with which the ACEs are associated, as well utility in the screening and discovery of drugs that modulate the ACE activity related to the disease.

In another embodiment, the invention provides methods for screening and identifying test compounds for their ability to modulate the activity of an individual ACE or a group or coordinated pattern of ACEs. In one embodiment, as discussed briefly above, two or more arrays can be prepared under similar conditions with one array acting as a control or reference for the other(s). For example, alteration of expression induced by a test compound such as a drug candidate may be determined by creating two arrays, one that corresponds to cells that have been treated with the test compound and a second that corresponds to the cells before treatment.

Differences in array data profiles can reveal which ACEs are affected by the test compound. An ACE may be more hypersensitive in the presence of the drug, as seen by more abundant hits at that ACE site during the nuclei incubation/reaction step leading to a stronger ACE signal in a profile. An ACE may be found less hypersensitive if, in comparison to a no drug control, a weaker signal were produced for that ACE spot in the array. In another example, an array profile obtained from a malignant tissue sample may be compared with an array profile obtained from a control or normal tissue sample. An inspection of the hypersensitive ACE differences between the arrays may reveal a genetic cause in the disease or a genetic factor in the disease progression.

In another embodiment, the arrays and methods of the invention are used for systematic and simultaneous identification of regulatory variants and their corresponding hypersensitivities (i.e. functional impact of variant). For example, this approach can be taken when a tissue containing a regulatory variant, such as a SNP, has been discovered it can be used to generate probes for screening by array profiling. If the position and nature of the regulatory variation is known relative to a nuclease cutting site, typically DNaseI, or to a restriction site, an indirect probe can be made from the tissue. The probe can be designed so as to contain the altered sequence. A collection of molecules could also be designed containing the versions of the regulatory sequence with and without the variation. The conditions of hybridization can be made so specific that matches between probes and targets only occur when they are homologous. In this way it can be shown whether a variation, which may occur as a heterozygous state, led to the failure of hypersensitive site formation. In still further embodiments, ACE regulatory variants can be screened, for example, for association with a particular disease state, for altered responsiveness to one or more test compounds relative to the corresponding wild type ACE sequence, and/or for association of a particular pharmacogenetic variant with a particular array signature.

In yet another embodiment, microarray based hybridization as described herein, or similar technologies available in the art, are used for the relatively high resolution profiling of a discrete genetic locus. For example, one can design oligonucleotides and primers to generate uniformly sized PCR products, which can be used to create collections of sequences which when either arrayed on a microarray, or some similar platform, allow the screening of contiguous or overlapping stretches of sequences covering genomic locations, e.g., a genetic locus of interest. Typically the genomic locations are chosen to include a gene locus, that is the entire sequence of a gene of interest and surrounding sequences in which it is likely that some or all of the regulatory elements of that gene are included. The amount of sequence covered on a single slide depends on a number of factors, but where necessary multiple slides can be used so there is no theoretical limit to the extent of sequences queried in this manner.

The length of the target DNA (the DNA that is immobilized) can vary from as small as 20 nucleotide of unique sequence in an oligonucleotide, though 35 or 60 nucleotides are more common. When oligonucleotides are used sequences are chosen which represent both strands of the DNA. PCR primers can also be designed to generate typically 250 bp or 500 bp products as target molecules. The sequences are generally designed so that they are either contiguous or adjacent molecules have some extent of overlap, the most extreme example of which is where with the oligonucleotide targets each sequence is shifted by a single base pair. Certain sequences, such as highly repetitive sequences, can be excluded from the target sequences. The platform selected-in the certain embodiments will be those in which the area of the microarray and the maximum number of spots it is possible to array.

In another embodiment, the arrays and methods of the invention are used for phylogenetic regulatory profiling. A large number of functionally active genetic elements would be expected to be conserved between different species, the more the closer the species are in evolutionary terms. Thus, according to another embodiment, probing a collection of these elements identified in one species, such as human, with a probe population constructed from a second species, such as mouse, would identify which of the elements have homologues in the probing population. This analysis of homologues can be extended to other species and also by comparing, amongst other attributes, the patterns of regulation of the homologues by creating probes from permissive and non-permissive tissues. These approaches have the advantage that nothing need be known about the genomic sequence of the organism from which the probe population is being made. Other methods rely on obtaining large amounts of sequence with which to perform multiple alignments in order to detect regions of conserved DNA, the biological activity of which then needs to be defined in a separate assay (conservation of sequence per se is not a foolproof marker of activity).

In another embodiment, ACE isolation and profiling in accordance with the present invention is amenable to array-based analysis for use in the discovery and analysis of underlying networks of genetic regulation. The use of such data is advantageous compared to cDNA expression data as the present methods enable monitoring the event or events which determine expression and, moreover, allows for analysis of large numbers of data points in an efficient and high throughput fashion.

In another embodiment, the methods and arrays described herein are used in the context of chemogenomic profiling. Chemogenomics represents the discovery and description of all possible compounds that can interact with any protein encoded by the human genome. Broadly, it now appears to mean taking a combinatorial approach to screening protein targets by family class and as such represent s a vast collection of closely related compounds which need to be screened in a high-throughput mode. Thus in another embodiment, ACE arrays described herein may be used to both confirm the pathway of action of any active molecule and to potentially detect any unexpected changes induced in the array.

In one specific embodiment of chemogenomic profiling, probes are prepared by cleaving genomic DNA with a chemotherapeutic agent, and profiles are thus established for different chemotherapeutic agents or different cells. It is known in the art that different cancers sometimes respond quite differently to a chemotherapeutic drug. Chemogenomic profiling of the response of different cancers to different chemotherapeutic agents permits the identification of cancers that may be more or less amenable to treatment by any given chemotherapeutic agent and can therefore be used to screen patients prior to treatment. For example, genomic sites targeted by a particular drug and associated with a favorable clinical outcome may be identified and then used to screen patients before treatment with the drug or to identify other cancers that may be amenable to treatment with the drug, since such cancers may display a similar chemogenomic profile. Furthermore, chemogenomic profiling according to the invention allows the identification of genomic locations that are modified in different tumors or by different drugs, as indicated by their particular profile. More specifically, insight may be gained into the disease process or the mechanism of action of the drug by examining chemogenomic profiles generated according to the invention. For example, profiles for a particular cancer may be examined before and after treatment with a drug known to be therapeutically effective to identify genomic locations that are modified in the tumor. Such locations are likely involved in the disease process.

In another embodiment, the methods and arrays described herein are used in the context of methylgenomic profiling. For example, probes are developed which are sensitive to, in the first instance, the presence of cytosine methylation in the CpG dinucleotide. It is known that this modification plays a role in genomic regulation. Other modifications can also be targeted with this technology and would include adenine methylation in plants or other organisms where it is found to occur and cytosine methylation where it occurs in different sequences, an example of which is C^{m}CWGG. Probing can be performed on a collection of sites, such as those contained in an array according to the present invention, or a locus profile, to for example examine changes in methylation patterns on induction of a gene, or on a genomic level, using a panel of microarrays or similar platform.

In yet another embodiment, the arrays and methods of the present invention may be used to evaluate deletions in genomic regulatory sequences. Two illustrative approaches are briefly described that can address this important question of how the loss of genetic material is associated with the onset of disease. For example, arrays described according to the present invention can be probed with a genomic DNA sample prepared from a diseased cell line or tissue and compared with a similar genomic reference probe (labeled with a different color) to determine and identify the ACE sequences that are either absent, or over represented, in the diseased state.. This strategy of using ACEs as genetic markers for this type of analysis offers the advantage over other approaches of identifying sequences which are most likely to be important in genomic regulation. In another example, one can generating probes from genomic DNA which map the occurrence of certain restriction sites. That is by use of cutters such as SseI8387I which on average cuts every 30 kb within the human genome to create indirect probe populations it is possible to perform hybridization with a custom tiling array containing all the sequence information immediately adjacent to this site. Spots on the array which show a change in signal, relative to a non diseased genomic probe created in a similar fashion, can be taken to represent where a change in the copy number of that particular restriction fragment has taken place in the diseased genome. Using this approach, it will be possible to estimate whether a deletion event is either hetero- or homozygous and also to determine the numbers of any duplication event. The choice of enzyme, its cutting frequency and properties (some enzymes show methylation sensitivity) will determine the resolution at which these genomic alterations can be mapped.

In another embodiment, the invention provides methods for comprehensively assessing the epigenetic status of chromatin in a sample by multimodality probing of array regulatory sequences. For example, the Chromatin Immunoprecipitation assay allows the recovery of DNA sequences from eukaryotic nuclei by antibody recognition of epitopes present on associated proteins within the nucleoprotein complex. This approach advantageously provides a means to recover DNA on the basis of either the enzymatic modifications of the histone proteins (referred to as the histone code and including, but not limited to, histone H4 and H3 acetylation, histone H3 methylation,and histone H1 phosphorylation) or the presence of specific proteins (be they members of the basal transcriptional machinery or certain transcription factors) or post-translationally modified versions of such proteins (which can be modified in a similar way to histone proteins). Once antibody recognition has been used to isolate the nucleoprotein complex the recovered DNA can be used to make one or more classes of probes, such as those described herein, e.g., pull-down probes, direct monotag probes or following restriction an indirect monotag probe.

Hybridization experiments useful in accordance with this embodiment may include the following. In one example, ChIp pull-down probes will be used to query a standard array spanning some genomic sequences, typically contiguous 250 bp fragments spanning 50-100 kb of a gene locus, in order to determine the patterns of an epigenetic modification and correlate it with previously determined expression and structural data. In another example, a reiteration of the above experiment is carried out with DNA prepared by performing the Chip experiments with a comprehensive collection of antibodies with specificity for all known and some novel histone modifications in order to generate a detailed description of the ‘histone code’ across a locus. In another example, by preparation of the ChIp-material from a range of transcriptionally permissive and non-permissive cells and tissues or following the effects of the histone code following environmental stimuli or induction of the gene with specific chemicals, it is possible to deduce the in vivo sequence of events which control or contribute to transcriptional regulation. Finally, another example involves assaying the effect of a class of potentially therapeutic molecules which are designed to modify the activities of the histone modifying enzymes not only on a gene of interest (as with locus profiling) but also by scanning large sections of the genome by creating in parallel an indirect monotag probe and hybridizing to appropriate tiling arrays.

In another embodiment, multimodality profiling is provided as an alternative to performing sequential screens with DNA reagents prepared by one of the discussed selection techniques (such as sensitivity to nucleases or chemicals, selection of nucleoprotein complexes by antibodies etc.). For example, one such approach can involve performing multiple selections in parallel, for example perform a Chip protocol with an antibody raised against histone H4 acetylation and then reselecting that population with a second antibody raised against a different modification. Similar combinations of Chip selections with nuclease/chemical sensitivity selections can be performed, as can selection based upon the methylation status of any preselected population.

The following specific examples are provided to illustrate embodiments of the invention, and should not be viewed as limiting the scope of the invention.

### EXAMPLES

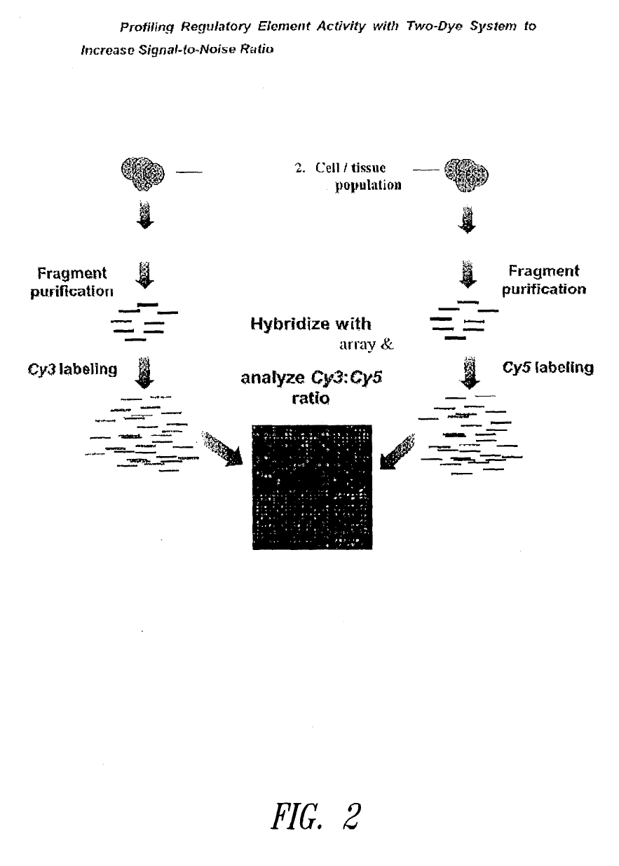

Many of the exemplified processes utilize combinations of new and old techniques and yield libraries of sub-cloned DNA fragments containing nuclease hypersensitive sites, as exemplified in FIGS. 1 and 2. A more specific example, as represented below and illustrated in FIG. 2 is a method that generates libraries of sub-cloned DNA fragments representing the complement of nuclease hypersensitive sites present in the chromatin of cells from erythroid cell lines.

Examples 1-3 set forth a general, but preferred, method for producing a hypersensitive site library from cultured hematopoetic cell lines. This method embodies the process illustrated in FIG. 2.

### EXAMPLE 1

### Preparation of DNA Microarrays Containing ACEs

Primer pairs were designed to allow amplification of approximately 500 bp PCR products from human genomic DNA. Following two rounds of amplification, where in the second one-hundredth volume of the original PCR reaction is used as a template, the PCR products are purified (using Millipore Multi-screen PCR purification plates), quantified (A260) and their concentration established to be between 50 ng/l-150 ng/ul. The size of the PCR products is checked by agarose gel eletrophoresis before the microarrays are printed (in 50% DMSO) onto mirrored slides (RPK0331, Amersham) using Amersham's Lucidea Arrayer. The PCR products are crosslinked to the slides with 500mJ, using Stratagene's Stratalinker. The slides are stored desiccated until use.

### EXAMPLE 2

### Preparation of DNA that Contains One or More Single-Stranded or Double-Stranded Cleavage Sites within Domains Defined by Aces.

K562 cells were grown to confluence (5×105 cells per cubit milliliter as assayed by hemocytometer). Nuclei were prepared from a suitable volume (e.g., 100 ml) and nuclei were prepared as described (Reitman et al MCB 13:3990). Briefly, Nuclei were resuspended at a concentration of 8 OD/ml with 10 microliters of 2 U/microliter DNaseI [Sigma] at 37° C. for 3 min. The DNA was purified by phenol-chloroform extractions and ethanol precipitated. The DNA was repaired in a 100 microliter reaction containing 10 microgram DNA and 6 U T4 DNA polymerase (New England Biolabs) in the manufacturer's recommended buffer and incubated for 15 min at 37° C. and then 15 min at 70° C. 1.5 U Taq polymerase (Roche) was added and the incubation continued at 72° C. for a further 10 min. The DNA was recovered using a Qiagen PCR Clean-up Kit and the DNA eluted in 50 microliter of 10 mM Tris.HCI, pH8.0

### EXAMPLE 3

### Isolation of DNA Fragments Associated with Aces.

DNA was mixed in a 100 microliter reaction volume containing 50 pmol of PS003 adapter (created by annealing equimolar amounts of oligonucleotides 5′ biotinylated PS003f and 5′ phosphorylated PS003r, to create an adapter containing a Noti site) and 40 U T4 DNA ligase (New England Biolabs) in the manufacturer's recommended buffer for 16 h at 4° C. The sequences of these oligonucleotides are: 5′ Bio_TTATGCGGCCGCTATGTGTGCAGT PS003F and 3′ GAATACGCCGGCGATACACACGTC PS003R.

The reaction was incubated at 65° C. for 20 min before the DNA was isopropanol precipitated in the presence of 0.3 M NaOAc and after ethanol washing resuspended in 20 microliter TE buffer (10 mM Tris.HCI, 1 mM EDTA, pH8.0). The DNA was digested in a 50 microliter reaction volume containing 20 U Hsp92 II (Promega) in the manufacturer's recommended buffer by incubation at 37° C. for 2 h, after which a further 20 U of enzyme was added and the incubation continued for 1 h and then heated to 72° C. for 15 min. The DNA was captured on M-270 Dynal beads as per manufacturer's instructions.

The beads were finally washed in 200 microliter of ligation buffer before capture and resuspension in a 100 microliter reaction volume containing 50 pmol of Hsp adapter (made by annealing equimolar amounts of oligonucleotides fHsp and rHsp) supplemented with 6 U T4 DNA ligase (New England Biolabs) in the manufacturer's recommended buffer and incubated at 16° C. for 16 h. The reaction was heated to 65° C. for 15 min prior to capture of the beads. The beads were washed in 1×NEB3 buffer (New England Biolabs) and then resuspended in a reaction volume of 100 microliter of the same buffer supplemented with 40 U Notl (New England Biolabs) and incubated for 37° C. for 1 hour with occasional mixing. Afterwards, the beads were captured and the supernatant retained. The beads were washed once and the resultant supernatant combined with the first and isopropanol precipitated in the presence of 20 microgram glycogen and 0.3 M NaOAc. After ethanol washing, the DNA was resuspended in 10 microliter of 10 mM Tris.HCI, pH8.0.

It will be clear to those skilled in the art that fragments isolated by the procedure above, or modifications thereof, may be used as reagents for the isolation or identification of genomic DNA segments that flank the site of DNA modification by combination with separately prepared population of genomic DNA that has been fragmented by other methods.

In the case of this specific embodiment/example, it is desirable to perform an amplification step prior to subcloning. It is anticipated that such a step may be required in some, but by no means all instances of the application of the process of the invention, as mentioned above. To perform amplification of the recovered DNA fragments prior to cloning, PCR may be employed or other methods of amplification, such as RCA (Rolling Circle Amplification) or versions of it. To render the fragments fit for PCR for example, another linker can be incorporated at the opposite end from that of the biotinylated linker mentioned above. A PCR amplification is then carried out.

To confirm that the DNA segments isolated with the above procedure contain ACE regions that would be expected in an erythroid cell line such as K562, the products are probed for the presence of nuclease ACEs known to be present in this cell type.

### EXAMPLE 4

### Labeling of DNA Fragments Associated with Aces

Two μ Fog of DNA were diluted into a volume of 24 μl with water and 20 μl of 2.5× Random Primers Solution (Invitrogen, constituent of BioPrime Labeling Kit) and the mixture heated to 95° C. for 5 min. The mixture is cooled on ice for 5 min before 2 ml dNTP solution (consisting of 5 mM Promega's dATP, dGTP, dTTP and 1 mM dCTP) and 3 μl of either 1 mM dCTP-Cy3 or dCTP-Cy5 (Amersham) and 1 μl of 40 U/ml Klenow (Invitrogen). The mixture was incubated at 37° C. for 2.5 h before being stopped by the addition of 5 μl of 0.5 M EDTA. The probes were purified on Qiagen QlAquick columns and eluted in 100 μl of EB. The amount of incorporation was calculated by reading the absorbance at 550 nm (for Cy3) and 650 nm (for Cy5) and probes were mixed at a dye molar ratio of 4:1 (pmol Cy3:pmol Cy5). Typically 200 pmol of Cy3 labeled probe was used and 50 pmol Cy5.

### EXAMPLE 5

### Preparation and Labeling of Control DNA Fragments

Genomic DNA was isolated from K562 nuclei which had not been treated with a nuclease (1 ml of nuclei with an A₂₆₀ of 8 OD/ml) and had been subsequently digested with NlaIII to completion and the DNA purified using a Qiagen Dneasy column. The concentration of the DNA was corrected to 150 ng/μl. These probes were labeled with Cy3.

### EXAMPLE 6

### Hybridization of Ace-Associated and Control DNA Fragments to Ace-Containing DNA Microarrays

The calculated amounts of probes were mixed and dried down in the dark. The paired probes are resuspended thoroughly in 8.5 μl 4× Hybridization buffer (Amersham, #RPK0325) and 8.5 μl water and then mixed with 17 μl formamide and vortexed. The mixture is heated at 95° C. for 3 min then cooled by spinning at 13K for 2 min. 30 μl of this hybridization solution was dispensed in a thin line across a slide and spread evenly over the surface by laying on of a coverslip and incubated at 42° C. for 16 h in a humid and darkened hybridization chamber.

The slides are washed in the dark with gentle agitation. The washes used were 5 min at 37° C. in Wash 1 (1×SSC, 0.2% SDS), two 5 min washes at 37° C. in Wash 2 (0.1×SSC, 0.2% SDS) and two 5 min washes at room temperature in Wash 3 (0.1×SSC). The slides were air-dried and scanned immediately using Packard Biosciences ScanArray 4000.

### EXAMPLE 7

### Overview of Processes

An overview of a representative process is illustrated in FIG. 1. This figure shows how the structural integrity of ACEs within a sample may be determined in a two step process: A probing reagent is created and compared to a query population. To create the reagent, cells are treated by a procedure developed to isolate and label a population of DNA fragments from the genome that is enriched in those structurally formed ACEs or a functional subset of them, such as transcriptional promoters, or a structural subset, such as methylated sequences. In this example, these DNA fragments are used as a probe to hybridize against a population of sequences on a microarray. Those sequences may be a set of previously characterized ACEs, may physically span a section of the genome or be a large enough combination of oligonucleotides to allow discretion of complex binding patterns. Following analysis the presence and intensity of the signal reflects the extent to which that particular ACE has formed within that population of cells.

Alternatively, the process may be carried out in parallel using two different markers in order to reveal a differential expression pattern. This process may be employed to increase the signal-to-noise ratio as illustrated in FIG. 2. Here, the sensitivity and accuracy of microarray hybridization will be maximized by comparing the signal of two populations of probes generated by the same procedure but isolated from a treated and non-treated population. In this example, the probe labeled with Cy3 is enriched for ACEs whilst the Cy5-labeled probe will contain ACEs at the same frequency as they occur in the genome. As the probes are generated the same way, they will share similar physical characteristics, such as length and labeling efficiency. Therefore, the ratio of intensity seen on a co-ordinate in the array will accurately reflect enrichment of the sequence in one of the probing populations. In this example, a structurally formed ACE in the cell population would give rise to a green (Cy3) spot, while an unformed site would be yellow (equal amounts of Cy3 and Cy5 bound) or red (Cy5).

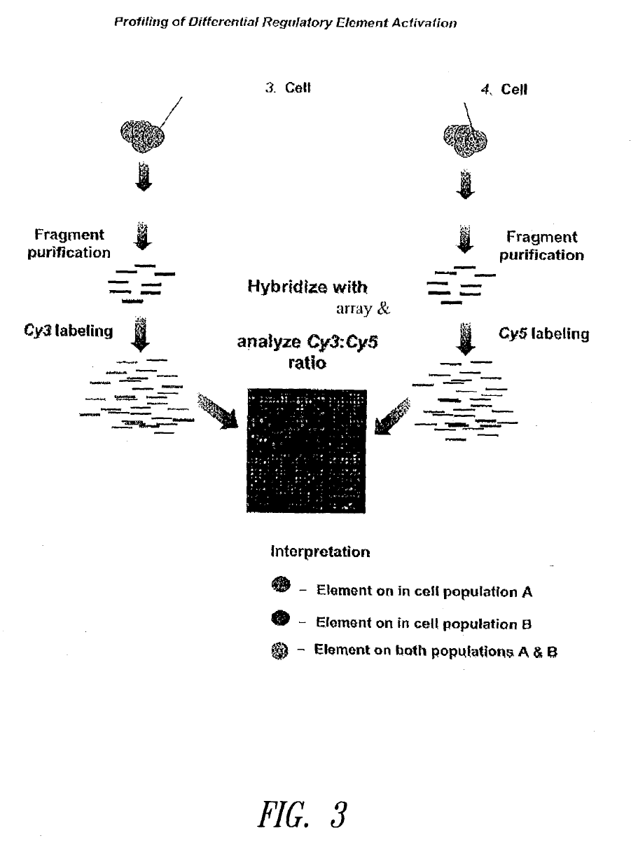

Several further additional applications of the invention are illustrated in FIGS. 3 through 6. These include:

i. Differential profiling of regulatory elements (i.e., between two different cell populations). An overview of this process is illustrated in FIG. 3. FIG. 3 shows how the technology can be used to examine the dynamic nature of ACE formation. In this example, two cell types are treated with a similar procedure to generate from each a differently labeled probe population enriched in ACEs. As in FIG. 2, the probes will have similar physical characteristics which allows their direct comparison. Hence, an ACE formed in one tissue but not the other will label its spot predominately red or green, while those formed in both tissues will color yellow. The exact ratio of Cy3 to Cy5 will provide information about the relative abundance of that ACE in the tissues. Any ACEs that are absent from both tissues will not be lit up on the array.

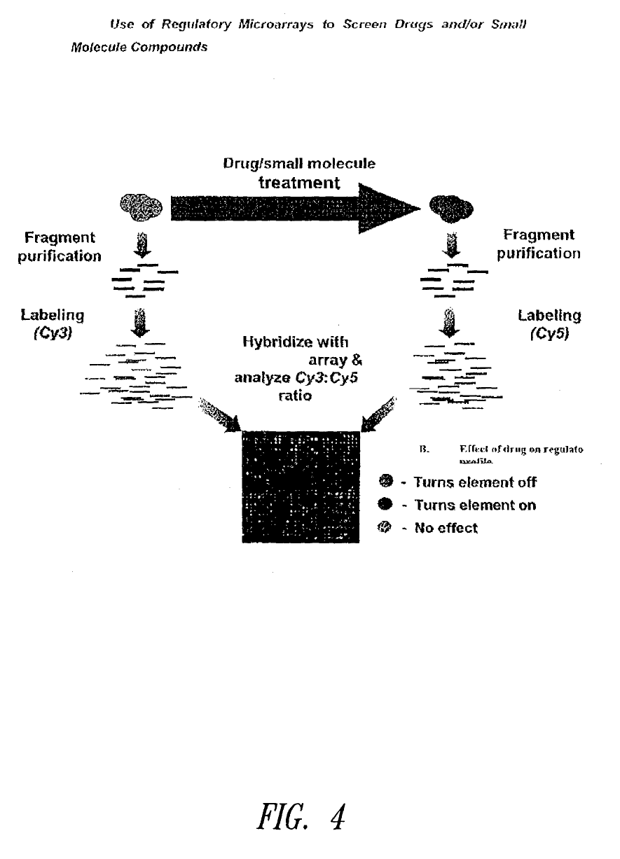

ii. Screening for compounds or treatments that impact the regulatory element activity profile. An overview of this process is illustrated in FIG. 4. As seen here, profile changes may be monitored to show changes in the pattern of ACE in response to stimuli. Comparative hybridization, as described in FIG. 3, can be used to determine, in this example, which ACEs are induced or repressed by treatment with a drug or small molecule. A probe population is prepared from a reference population of untrerated cells and compared to that of a differently labeled probe from the cells following treatment following hybridization to the microarray.

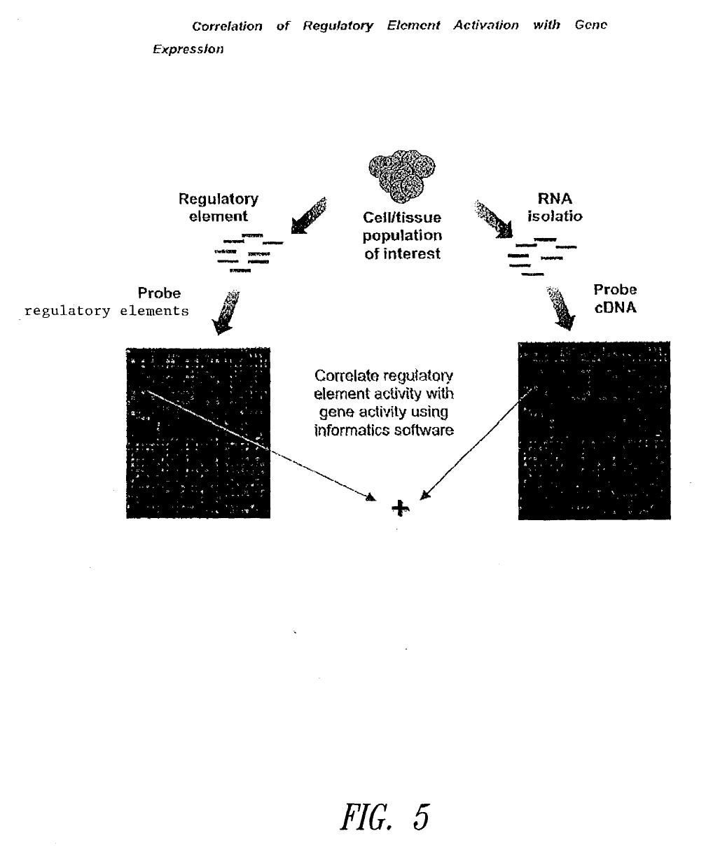

iii. Correlation of regulatory element activation patterns with gene expression patterns to construct regulatory network maps. An overview of this process is illustrated in FIG. 5, which establishes a correlation between ACE and expression data. Parallel analysis of gene expression, as detected by use of expression arrays, and ACE structural integrity will give information about ACEs implicated in transcriptional control of specific genes. Such correlation will also enable improved quality control for conventional expression arrays.

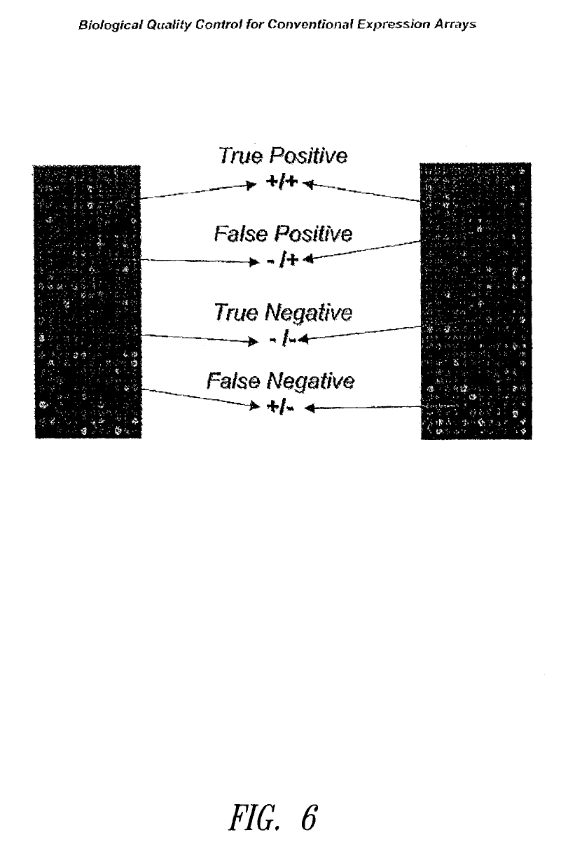

iv. Correlation of regulatory element activation with gene expression to provide a powerful biological quality control assay for gene expression arrays. An overview of this process is illustrated in FIG. 6.

### EXAMPLE 8

### Illustrative Method for the Production of Fixed Length, Direct Monotag Probes for Hybridization to Ace Microarrays

This example describes an illustrative procedure for use in generating direct monotag probes for use in accordance with the present invention.

A. Genomic DNA is First Cleaned Using a Centricon YM30 Column, as Follows:

1. Wash Centricon 30 column through with 400ul TE pH 8.0 or water

2. Spin 10 mins @ 6000 rcf

3. Add g.DNA (10-15 ug) and spin 10 mins @ 6000 rcf

4. Wash 2×500 ul TE pH 8.0 and spin 15 mins each

5. Elute with 200 ul TE (10Mm Tris 0.2Mm EDTA)

6. Let column sit 30 mins @ 37° C.

7. Invert column and spin 3000 rcf for 3 min

8. Check DNA on 0.8% agarose gel and take OD.

B. Blunting and Tailing of the DNA is Performed as Follows:

1. Combine 100 ul cleaned gDNA & 11.0 ul 10×PCR buffer+MgCl₂

2. Incubate @ 65′C for 10 mins

3. Place on ice and add Master Mix

4. Prepare Tailing Mix as follows:

4.0 ul 10×PCR buffer×MgCl2

2.0 ul dNTP's 10Mm

1.0 ul T4 DNA polymerase

1.0 ul Taq polymerase

30.0 ul H20

5. Add 40.0 ul tailing mix to DNA and incubate @ 37′C for 15 mins

6. Remove and incubate @ 72′C to add A's for 15 mins

7. Clean on PCR clean-up column to remove enzymes. etc.

8. Elute in 150.0 ul EB

C. Ligation of Adapter 1 is Performed as Follows:

5′Biotin-CTC TGG CGC GCC GTC CTC TCA CGC GTC CGA
CT GAG ACC GCG CGG CAG GAG AGT GCG CAG GCT G-5′ P

1. Prepare Ligation Mix as follows:

143 ul cleaned gDNA

16 ul 10×ligase buffer

1.0 ul Adapter 1 @ 50pmol/ul

*0.5 ul T4 DNA ligase NEB 400U/ul

2. Add ligase in 1×ligase buffer+0.5 ul ligase 10 ul per tube

D. Cleaning Up O/N Ligation to Remove Un-Incorporated Adapter is Performed as Follows:

1. Clean using PCR column as per manufacturer's instructions (Qiagen)

2. Elute with 500 ul TE preheated to 55′C

3. Leave for 10 mins at 37′C

4. Spin and retain 1.0 ul to run on QC gel

5. Clean again using Centricon 100 column—prepare column as before by eluting through with 400 ul TE/water to remove glycerol.

6. Spin at 200 rcf

7. Load on elute from PCR column (500 ul)

8. Spin at 500 rcf for 15 mins (retain elute)

9. Wash ×2 500 ul TE and spin again at 500 rcf for 15 mins (filter should look fairly dry at this point)

10. Add 100 ul of 10 Mm Tris Ph 8.0

11. Allow to sit 30 min to re-dissolve DNA bound to column

12. Carefully invert column and collect in clean tube by spinning at 3000 rcf for 3 min

13. Run 5.0 ul of first flow through and 1.0 ul of collected sample on QC gel (0.8% Agarose)

14. Run for 60 min, stain and scan.

E. Digest 1 with Mme 1

1. Prepare digestion mixture as follows:

100 ul Adapter DNA

11.5 ul 10×MmeI buffer

1.0 ul SAM at 50 uM final conc.

2.0 ul MmeI

1.0 ul BSA

F. Binding to Beads

1. Re-suspend 10 ul M271 and capture

2. Wash×2 in 1×BB

3. Re-suspend in 115 ul 2×BB and add beads to MmeI digested DNA

4. Allow to bind at room temperature on rocker for 30 mins

5. Capture and retain s/nat for QC gel

6. Wash×2 in wash buffer (10 Mm Tris pH8.0, 50 Mm Nacl, 1Mm EDTA)

G. Digest 2 with MmeI

1. Wash in 50 ul 1×MmeI buffer

2. Capture and re-suspend in 30 ul digest

3.0 ul 10×NEB4 buffer

3.0 ul SAM (1/64 dil)

22.0 ul H20

2.0 ul MmeI

0.5 ul BSA

3. Digest for another 30 mins at 37° C.

4. Capture on beads and repeat digestion once more by re-suspending beads in digestion mix

5. Incubate 37° C. for another 30-40 mins

H. Labelling Monotags

1. The beads are then used directly in a labelling reaction using an oligo labelled with Cy5 or Cy3. 5′Cy5/3-CTC TGG CGC GCC GTC CTC TCA CGC GTC CGA CT

2. The following mixture is added to 1 μl of the beads:

10 ul PCR buffer

4.0 ul labelled oligo (5 pmol/μl)

2.0 ul 10 mM dNTPs

0.5 ul hot start Taq

83.5 ul water

3. The reaction mixture is cycled on the following program: 95° C. for 2 min, 93° C. for 15 s, 60° C. for 15 s, 72° C. for 15s; ×30; 72° C. for 2 min, 4° C. on hold

### EXAMPLE 9

### Illustrative Method for the Production of Fixes Length, Indirect Monotag Probes for Hybridization to Ace Microarrays

A. Digestion of Genomic DNA with Sse8387I

Sse8387I is an 8-cutter enzyme, insensitive to methylation, which recognizes and restricts the site 5′-CCTGCA↓GG-3′ and has an estimated 10⁵ sites in the human genome is used as follows.

1. Digest two aliquots of 20 μg each of clean genomic DNA from either a cell line (K562) or primary tissue

2. Phenol-chloroform extract

3. Ethanol precipitate in the presence of 1/10 volume of 3 M NaOAc and 2 volumes ethanol

4. Wash and resuspend in 10 μl water

B. Ligation of Linkers

1. The following oligonucleotides are annealed to give two sets of linkers:

PS_Af (5′ Biotin)
CTC TGG CGC GCC GTC CTC TCA CGC GTC CGA CTG CA
PS_Ar (5′ Phosphate)
GTC GGA CGC GTG AGA GGA CGG CGC GCC AGA GC
PS_A Linker
5′-Biotin MluI MmeI
CTC TGG CGC GCC GTC CTC TCA CGC GTC CGA CTG CA
C GAG ACC GCG CGG CAG GAG AGT GCG CAG GCT G

2. Set up the following two ligations:

4 μl 10×T4 DNA ligase buffer (Promega);

1 μl T4 DNA ligase (3U/ml);

10 μl Sse8387I-digested DNA (10 μg);

1 μl PS_Linker A or B (50 pmol/μl);

24 μl water.

3. Incubate overnight at 4° C.

4. Clean reaction on DNeasy column to remove unincorporated primers

5. Resuspend in 10 μl EB buffer

6. Ethanol precipitate in the presence of 1/10 volume of 3 M NaOAc and 2 volumes ethanol

7. Wash and resuspend in 10 μl water.

C. Digestion with MmeI

1. Set up the following digestions on both samples:

3 μl 10×MmeI buffer (Gdansk);

10 μl Sse8387I-digested DNA+Linker A (10 μg);

1 μl MmeI (2 U/μl);

16 μl water.

2. Incubate at 37° C. for 3 hours

3. Capture on M-270 Dynal beads

4. Wash 10 μl Dynal beads twice with 100 μl 2×Binding buffer, resuspend beads in 30 μl 2×Binding buffer and combine with 30 μl of MmeI-digests. Allow to bind for 30 mins at room temperature with mixing

D. Labelling Monotags

1. The beads are then used directly in a labelling reaction using an oligo labelled with Cy5 or Cy3 5′Cy5/3-CTC TGG CGC GCC GTC CTC TCA CGC GTC CGA CTG CA

2. The following mixture is added to 1 μl of the beads:

10 ul PCR buffer

4.0 ul labelled oligo (5 pmol/μl)

2.0 ul 10mM dNTPs

0.5 ul hot start Taq

83.5 ul water

3. The reaction is cycled on the following program: 95° C. for 2 min, 93° C. for 15 s, 60° C. for 15 s, 72° C. for 15s; ×30; 72° C. for 2 min, 4° C. on hold

### EXAMPLE 10

### Illustrative Method for the Production of Variable Length, Direct Pull Down Probes for Hybridization to Ace Microarrays

The Cy5 probe was prepared as follows. Nuclei were prepared from K562 cells and resuspended at a concentration of 8 OD/ml with 10 μl 2 U/μl DNaseI [Sigma] at 37° C. for 3 min. The DNA was purified by phenol-chloroform extractions and ethanol precipitated. The DNA was repaired in a 100 μl reaction containing 10 μg DNA and 6 U T4 DNA polymerase (New England Biolabs) in the manufacturer's recommended buffer and incubated for 15 min at 37° C. and then 15 min at 70° C. 1.5 U Taq polymerase (Roche) was added and the incubation continued at 72° C. for a further 10 min. The DNA was recovered using a Qiagen PCR Clean-up Kit and the DNA eluted in 50 μl of 10 mM Tris.HCI, pH8.0. The DNA was mixed in a 100 μl reaction volume containing 50 pmol of adapter A (created by annealing equimolar amounts of oligonucleotides 5′ biotinylated PSAf and 5′ phosphorylated PSAr) and 40 U T4 DNA ligase (New England Biolabs) in the manufacturer's recommended buffer for 16 h at 4° C. The reaction was incubated at 65° C. for 20 min before the DNA was isopropanol precipitated in the presence of 0.3 M NaOAc and 10 μg glycogen and after ethanol washing resuspended in 20 μl TE buffer (10 mM Tris.HCI, 1 mM EDTA, pH8.0). The DNA was digested in a 50 μl reaction volume containing 20 U Hsp92 II (Promega) in the manufacturer's recommended buffer by incubation at 37° C. for 2 h, afterwhich a further 20 U of enzyme was added and the incubation continued for 1 h and then heated to 72° C. for 15 min. The DNA was captured on M-270 Dynal beads as per manufacturer's instructions. The beads are then used directly in a labelling reaction using PSAf labelled with Cy5 or Cy3. The following PCR reaction is performed on the beads in a 100 ml volume containing 25 pmol labeled PSAf, 0.2 mM dNTPs and 2.5 U Taq polymerase. The mixture is cycled at 95° C. for 2 min, 93° C. for 15 s, 60° C. for 15 s, 72° C. for 15s; ×30; 72° C. for 2 min, 4° C. on hold.

### EXAMPLE 11

### Illustrative Method for the Production of Probes from Chromatin Fractions for use in Hybridization to Ace Microarrays

A. Isolation of Formaldehyde Crosslinked Chromatin Fragments

1. Start with nuclei isolated from K562 cells prepared according to the standard tissue preparation protocol. After the nuclei are pelleted they are washed and resuspended in PDS pH 7.4 with 1 mM EDTA and 0.5 mM EGTA and freshly added protease inhibitors.

2. Add formaldehyde to a final concentration of 0.5% and mix gently at room temperature for 10 min.

3. Quench crosslinking reaction by adding 2.5 M glycine to a final concentration of 125 mM. Stir at room temperature for an additional 5 min.

4. Pellet nuclei by spinning for 5 min at 1500 g at 4° and resuspend in the smallest amount of buffer possible. (Having the solution very concentrated here will reduce the need to concentrate it later. It seems that SDS is not required in this buffer as SDS does not lyse crosslinked cells, but sonication does. One dialysis step will be avoided if the sonication is performed in Xba Digest Buffer (XDB; 10 mM Tris pH 8.0, 1 mM MgCl₂, 50 mM NaCl, 1mM BME). Maintain conditions as cold as possible.

5. Sonicate to give DNA-protein complexes that have roughly 500 bp of DNA.

B. Digest DNA with Xbal and Exonuclease to give Single Stranded Regions for Binding of Biotinlyated Primers

1. If the sonication is performed in XDB, immediately add Xbai (10 U/ug DNA) to solution and incubate at 37°. It is preferred to minimize the time at 37°. For example, one can use a 3 hr digestion, adding the enzyme in two different aliquots 1.5 hr apart.

2. λ exonuclease may be added at a final concentration of 1 U/ug DNA directly to the Xba digest and incubated at 37° for 2 h. Quench the reaction with 1 mM EDTA.

C. Capture of Chromatin-Protein Complexes.

This is a two step process. First, biotinylated primers must bind to the HBB HS2 site, and second these biotinylated complexes must bind to Streptavidin-coated coated Dyna beads.

1. Dialyze into the solution hybridization buffer-perform dialysis at 40.

a) 10 mM Tris (8.0), 1 mM EDTA, 1 M NaCl,

b) 10 mM Tris (8.0), 1 mM EDTA, 1 M NaCl, 10% DMSO

2. Hybridize with biotinylated primers.

a. Add 6 biotinylated oligos spanning the HBB HS2 site at 3.6 nM each and heat sample to 80° for 10 min. and then cool slowly to 37°.

b. Incubate chromatin with biotinylated oligos at 42° C.

3. Capture complexes on Dyna M270 beads.

Other embodiments and uses of the invention will be apparent to those skilled in the art from consideration of the specification and practice of the invention disclosed herein. All references cited herein, including all U.S. and foreign patents and patent applications including U.S. Provisional patent No. 60/108,206, and U.S. patent application Ser. No. 09/432,576, are specifically and entirely hereby incorporated herein by reference. It is intended that the specification and examples be considered exemplary only, with the true scope and spirit of the invention indicated by the following claims.

1. A nucleic acid array comprising a plurality of active chromatin elements.

2. The array of claim 1 wherein each active chromatin element contains a nuclease hypersensitive site.

3. The array of claim 2 which further comprises one or more sets of nucleic acid sequences that tile across one or more hypersensitive sites.

4. The array of claim 3 wherein the one or more sets each comprise sequences within 200 nucleotides of said hypersensitive site.

5. The array of claim 1 wherein the plurality comprises sequences derived from an organism.

6. The array of claim 3 wherein the organism is selected from the group of organisms consisting of Homo sapien, rat, mouse, zebrafish, drosophila, yeast, C. elegans, and combinations thereof.

7. The array of claim 1 wherein the plurality comprises nucleic acid sequences with lengths from about 16 nucleotides to about 1,500 nucleotides.

8. The array of claim 1 wherein the plurality comprises nucleic acid sequences with lengths from about 100 nucleotides to about 350 nucleotides.

9. The array of claim 1 wherein the plurality comprises at least 100, at least 1,000, at least 10,000. at least 100,000, or at least 1,000,000 active chromatin elements.

10. The array of claim 1 which further contains nucleic acids that represent transcribed sequences.

11. The array of claim 1 which further contains sequences that flank active chromatin elements.

12. The array of claim 1 which further contains repetitive sequences.

13. The array of claim 12 wherein repetitive sequences comprise less than five percent of the total nucleic acids of the array.

14. The array of claim 1 prepared by a process comprised of treating cells with an agent that induces modifications in the nucleic acid.

15. The array of claim 14 wherein the modification is selected from the group consisting of cleavage, methylation, radiation, and combinations thereof.

16. The array of claim 14 wherein the modified nucleic acids are subtracted from nuclease treated unmodified nucleic acids.

17. The array of claim 14 further comprising the step of attaching biotin to the modified nucleic acids.

18. The array of claim 14 further comprising the step of amplifying the modified nucleic acid by PCR.

19. A method for forming the array of claim 1 comprising:
treating genomic DNA with an agent that induces modifications in said DNA;
treating a portion of the modified DNA with nuclease;
subtracting nuclease treated DNA from the modified DNA; and
obtaining an array of active chromatin elements.

20. The method of claim 19 wherein the modifications comprises cleavages that create DNA fragments.

21. The method of claim 20 wherein the DNA fragments are ligated to a linker.

22. The method of claim 21 wherein the linker-ligated DNA fragments are isolated.

23. The method of claim 20 wherein the fragments are cut into smaller sizes by a procedure selected from the group consisting of digestion with a restriction enzyme and sonication.

24. A method for determining the active chromatin element profile of nuclear chromatin of a cell comprising:
treating a portion of said chromatin with an agent that preferentially modifies DNA at hypersensitive sites to form a first set of nucleic acids;
treating another portion of said chromatin with another agent that non-preferentially modifies DNA to form a second set of nucleic acid; and
comparing the first and second sets to obtain said active chromatin element profile.

25. The method of claim 24 wherein the first and second sets are compared by hybridization.

26. The method of claim 25 wherein the first or second set is amplified by PCR.

27. The method of claim 25 wherein the first or second set is labeled with a fluorescent dye.

28. A method for identifying a profile of DNA regulatory elements in a eukaryotic cell comprising:
treating said cell with an agent that modifies DNA of said cell at DNA hypersensitive sites; and
identifying the DNA hypersensitive sites from said reaction with the agent, wherein the nucleotide sequences of said DNA hypersensitive sites and the locations thereof in the DNA of said type of cells constitute a profile of DNA regulatory elements in said type of cells.

29. A method for producing a profile of DNA regulatory elements in eukaryotic cells, comprising:
treating said cells with an agent that modifies eukaryotic DNA at DNA hypersensitive sites;
identifying the DNA hypersensitive sites from said reaction with said agent wherein the nucleotide sequences of said DNA hypersensitive sites and the locations thereof in the DNA of said type of cells constitute a profile of DNA regulatory elements in said type of cells; and
isolating the nucleotide sequences of said hypersensitive sites.

30. The method of claim 29 wherein one or more oligonucleotide linkers are ligated into said nucleotide sequences.

31. The method of claim 30 wherein said oligonucleotide linkers are biotinylated and wherein said isolating is performed using streptavidin-coated magnetic beads.

32. The method of claim 30 further comprising amplifying said nucleotide sequences by polymerase chain reaction.

33. The method of claim 29 wherein the eukaryotic cells are selected from the group consisting of primary cell cultures, cell lines, newly isolated cells from an organism, and combinations thereof.

34. The method of claim 29 wherein the eukaryotic cells are normal cells or abnormal cells.

35. The method of claim 34 wherein the abnormal cells are cancer cells.

36. The method of claim 29 wherein said agent is selected from the group consisting of radiation, a chemical agent, an enzyme, and combinations thereof.

37. The method of claim 36 wherein the radiation comprises UV light radiation.

38. The method of claim 36 wherein the chemical agent is a clastogen.

39. The method of claim 36 wherein the enzyme is selected from the group consisting of specific endonucleases, non-specific endonucleases, topoisomerases, methylases, histone acetylases, histone deacetylases, and combinations thereof.

40. The method of claim 39 wherein the specific endonuclease comprises one or more four-base restriction endonucleases, one or more six-base restriction endonucleases, or combinations thereof.

41. The method of claim 40 wherein the four-base restriction endonuclease is selected from the group consisting of Sau3a, Styl, NlaIII, Hsp 92, and combinations thereof.

42. The method of claim 40 wherein the six-base endonuclease is selected from the group consisting of EcoRl, HindIII, and combinations thereof.

43. The method of claim 39 wherein the non-specific endonuclease is DNase I.

44. The method of claim 39 wherein the topoisomerase is topoisomerase II.

45. A profile of DNA regulatory elements in eukaryotic cells as produced by the method of claim 29, said profile comprising isolated nucleotide sequences of the hypersensitive sites.

46. The profile of claim 45 wherein the eukaryotic cells are selected from the group consisting of primary cell cultures, cell lines, newly isolated cells from an eukaryotic species, and combinations thereof.

47. The profile of claim 46 wherein the eukaryotic cells are normal cells or abnormal cells.

48. The profile of claim 47 wherein said abnormal cells are cancer cells.

49. The profile of claim 45 wherein the nucleotide sequences are labeled with a fluorescent dye, a radioactive nucleotide, a magnetic particle, or a combination thereof.

50. A nucleotide array having spotted thereon the profile of claim 45.

51. The nucleotide array of claim 50 wherein the array is fixed to a slide, a chip, or a membrane filter.

52. The nucleotide array of claim 50 wherein one or more copies of said nucleotide sequences of the hypersensitive sites are spotted on said array.

53. A method for detecting DNA regulatory elements in eukaryotic cells comprising:
a) isolating mRNAs from said cells, converting said mRNA's to cDNA and probing an array to generate a profile;
b) isolating active regulatory elements from said cells and probing an array to generate a profile, and
c) comparing the profile from the cDNA probe with the profile from the active regulatory elements probe to correlate regulatory element activity with gene activity.

54. A method for detecting DNA regulatory elements in eukaryotic cells comprising:
a) isolating mRNAs from the cells;
b) contacting said isolated mRNAs to the array of claim 1 to detect hybridization signals, wherein the nucleotide sequences of hybridized spots represent the DNA regulatory elements of said cells.

55. A sequence library of active chromatin elements encoding fragments suitable for preparing a profile to determine the regulatory status of a eukaryotic cell sample.

56. The library of claim 55 wherein the fragments are obtained by the step of marking hypersensitive sites of nuclei of the eukaryotic cells of the sample.

57. The library of claim 56 wherein the marking step is carried out by incubating DNAse I with the nuclei to form nicks in DNA at the hypersensitive sites.

58. The library of claim 55 wherein less than five percent of the fragments contain repetitive DNA sequences.

59. The library of claim 55 wherein each fragment comprises a first end generated by cleavage with DNase I and a second end generated by cleavage with another nuclease.

60. The library of claim 55 wherein the library exists in silico.

61. The library of claim 55 wherein the library exists in a vector.

62. The library of claim 61 wherein the vector is selected from the group consisting of microbial cell culture, plasmid vectors and eukaryote cell culture.

63. A library of active chromatin element primers, prepared by obtaining a library of active chromatin element fragments and determining sequences outside the active chromatin element fragments suitable for cloning the active chromatin element fragments.

64. The library of claim 63 which contains at least 10, at least 100, at least 1,000, at least 10,000, at least 100,000 or at least 1,000,000 active chromatin element primers.

65. The library of claim 63 wherein the library is in silico.

66. A method for profiling active chromatin elements from a sample that contains nucleic acid, comprising:
a) obtaining one or more purified or labeled active chromatin elements from the sample;
b) contacting the active chromatin elements from step a) with a DNA microarray containing DNA species in separate locations that match sites of the genome; and
c) detecting binding between the active chromatic elements and sites of the microarray.

67. The method of claim 66 wherein detecting comprises a detection system that involves fluorescence or chemiluminescence to determine position location in the array.

68. The method of claim 66 wherein the DNA microarray comprises immobilized oligonucleotide probes between 5 and 40 nucleotides in length occupying separate known sites of the array.

69. The method of claim 68 wherein the immobilized DNA oligonucleotide probes comprise at least two sets of probes wherein a first set that is exactly complementary to at least one reference sequence and comprises probes that span the reference sequence and which sequentially overlap each other, and at least one additional set of probes, each additional set of which is identical to the first set but for at least one different nucleotide, which different nucleotide is located in the same position in each additional set but which is a different nucleotide in each set.

70. The method of claim 68 wherein the immobilized DNA oligonucleotide probes comprise at least two sets of probes, a first set that is exactly complementary to at least one reference sequence and comprises probes that span the reference sequence and which overlap each other in sequence, and at least one additional set of probes, each additional set of which is identical to the first set but for at least one different nucleotide addition or deletion.

71. The method of claim 66 wherein the DNA species of the DNA microarray are genomic elements.

72. The method of claim 66 wherein the detected binding of step c) is recorded as a reference profile in a computer memory device.

73. A method of ascertaining the effect of an chemical or other environmental perturbation on a regulatory profile of a tissue obtained from a eukaryotic organism comprising;
a) obtaining a first profile for binding between active chromatic elements of the tissue that is unexposed to the perturbation and a microarray as described in any of claims 1 to 7;
b) obtaining a second profile for binding between active chromatic elements of the tissue and a microarray of claim 1 after exposure of the tissue to the perturbation; and
c) comparing the first profile with the second profile to determine genetic elements that are effected by the perturbation.

74. The method of claim 73 wherein the perturbation occurs before obtaining the tissue from the organism and wherein the environmental perturbation is selected from the group consisting of an infection of the eukaryotic organism from a microorganism, loss in immune function of the eukaryotic organism, exposure of the tissue to high temperature, exposure of the tissue to low temperature, cancer of the tissue, cancer of another tissue in the eukaryotic organism, irradiation of the tissue, exposure of the tissue to a chemical or other pharmaceutical compound, and aging.

75. The method of claim 73 wherein the perturbation occurs after obtaining the tissue from the organism and wherein the perturbation is selected from the group consisting of exposure of the tissue to high temperature, exposure of the tissue to low temperature, irradiation of the tissue, exposure of the tissue to a chemical or other pharmaceutical compound, and aging.

76. The method of claim 75 wherein the perturbation is the addition of one or more compounds.

77. The method of claim 76 further comprising the addition of at least one known pharmaceutical compound to the tissue prior to obtaining a profile for binding between active chromatic elements of the tissue and a microarray.

78. A method of discerning at least one set of co-regulated genes in cells of a eukaryotic organism, comprising:
obtaining a first profile for binding between active chromatic elements of the tissue under controlled culture conditions;
obtaining a second profile for binding between active chromatic elements of the tissue under conditions where a known regulator of at least one of the genes is altered with respect to the controlled culture conditions; and
comparing the first profile with the second profile from b) to determine which genetic elements are effected by the alteration of the known regulator.

79. The method of claim 78 wherein the regulator is a hormone, nutrient, or pharmacologically active chemical.

80. A nucleotide array having spotted thereon a set of nucleic acids between 5 and 75 nucleotides long obtained from the profile of claim 45.

81. The nucleotide array of claim 80, wherein said array is a slide, a chip, or a membrane filter.

82. The method of any of claims 19, 24, 28, or 29,, wherein the sample is selected from the group consisting of primary cell cultures, cell lines, newly isolated cells from an eukaryotic species, and combinations thereof.

83. A method for profiling active chromatin elements from a sample that contains nucleic acid, comprising:
a) obtaining one or more purified active chromatin elements from the sample and label them;
b) contacting the labeled active chromatin elements from step a) with a DNA microarray containing DNA species in separate locations that match putative or verified regulatory elements; and
c) detecting binding between the active chromatic elements and sites of the microarray.

84. The method of claim 83, wherein detecting comprises a detection system that involves fluorescence or chemiluminescence to determine binding.

85. The method of claim 83, wherein the DNA microarray comprises immobilized oligonucleotide probes between 5 and 40 nucleotides in length occupying separate known sites of the array.

86. The method of claim 85, wherein the immobilized DNA oligonucleotide probes comprise at least two sets of probes wherein a first set that is exactly complementary to at least one reference sequence and comprises probes that span the reference sequence and which sequentially overlap each other, and at least one additional set of probes, each additional set of which is identical to the first set but for at least one different nucleotide, which different nucleotide is located in the same position in each additional set but which is a different nucleotide in each set.

87. The method of claim 85, wherein the immobilized DNA oligonucleotide probes comprise at least two sets of probes, a first set that is exactly complementary to at least one reference sequence and comprises probes that span the reference sequence and which overlap each other in sequence, and at least one additional set of probes, each additional set of which is identical to the first set but for at least one different nucleotide addition or deletion.

88. The method of claim 83, wherein the DNA species of the DNA microarray are known regulatory sequences.

89. The method of claim 83, wherein the detected binding of step c) is recorded as a reference profile in a computer memory device.

90. A method for profiling active chromatin elements from a sample that contains nucleic acid, comprising:
a) obtaining multiple active chromatin elements from the sample and label them with a first label;
b) obtaining multiple genomic DNA fragments from the sample and label them with a second label;
c) hybridizing the elements from a) and the fragments from b) with a DNA microarray containing DNA species in separate locations that match putative or verified regulatory elements; and
d) determining the ratio of signals from the first and second labels within the array.

91. A method for profiling differential regulatory element activation from two populations that contain nucleic acid, comprising:
a) obtaining multiple active chromatin elements from the first population and labeling them with a first label;
b) obtaining multiple active chromatin elements from the second population and labeling them with a second label;
c) hybridizing the elements from a) and the fragments from b) with a DNA microarray containing DNA species in separate locations that match putative or verified regulatory elements; and
d) determining the ratio of signals from the first and second labels within the array.

92. The method of claim 91, wherein one of the populations is an untreated control, the other population is treated by contact with at least one chemical agent, and the signal ratios obtained in step d) provide an indication of gene regulatory activity by the at least one chemical agent.

93. The method of claim 91, wherein the signal ratios obtained in step d) indicate whether the at least one chemical agent turns on, turns off or has no effect on active chromatin elements.

94. A method for correlating regulatory element activation with gene expression from a sample that contains nucleic acid, comprising:
a) obtaining multiple active chromatin elements from the sample and profiling them on a DNA microarray containing DNA species in separate locations that match putative or verified regulatory elements;
b) isolating RNA from the sample and converting to cDNA;
c) profiling the cDNA on a DNA microarray containing DNA species in separate locations that match putative or verified regulatory elements; and
d) correlate the profile results from a) and c) with gene activity using informatics software.

95. A method of identifying an ACE profile associated with a disease state, comprising;
a) obtaining a first profile or set of profiles for binding between active chromatin elements of a tissue, said first profile or set of profiles being representative of a normal healthy condition;
b) obtaining a second profile or set of profiles for binding between active chromatin elements of a tissue, said second profile or set of profiles being representative of a disease condition; and
c) comparing the first profile or set of profiles with the second profile or set of profiles to identify alterations in the activity of one or more ACE elements in the disease condition relative to the normal condition.

96. A disease associated ACE profile or set of profiles identified according to the method of claim 95.

97. A method for diagnosing the presence of a disease condition in a patient, comprising obtaining an ACE profile for a biological sample obtained from a patient suspected of having said disease condition and comparing said ACE profile to a disease associated ACE profile or set of profiles according to claim 96.

98. The nucleic acid array of claim 1 wherein the active chromatin elements are associated with a particular cell type.

99. The nucleic acid array of claim 98 wherein the active chromatin elements are associated with a diseased cell.

100. A method for isolating ACE sequences in a eukaryotic cell comprising:
a) preparing nuclei from a biological sample;
b) treating the nuclei to form cross-linked chromatin-protein complexes;
c) treating the cross-linked chromatin-protein complexes to reduce the size of the DNA sequences associated with the complexes;
d) capturing the chromatin-protein complexes; and
e) isolating DNA sequences associated with the chromatin-protein complexes.

101. The method of claim 100, wherein the cross-linked chromatin-protein complexes are formed by treatment with a cross-linking agent.

102. The method of claim 101, wherein the cross-linked chromatin-protein complexes are formed by treatment with formaldehyde.

103. The method of claim 100, wherein the cross-linked chromatin-protein complexes are captured with an antibody.

104. The method of claim 103, wherein the antibody is specific for a protein of the cross-linked chromatin-protein complex.

105. The method of claim 104, wherein the antibody is specific for a histone protein.

106. The method of claim 104, wherein the antibody is specific for a member of the basal transcriptional machinery.

107. The method of claim 104, wherein the antibody is specific for a transcription factor.

108. The method of claim 100, wherein the cross-linked chromatin-protein complexes are captured with one or more oligonucleotide primers.

109. The method of claim 108, wherein the one or more oligonucleotide primers are designed to bind to an HBB HS2 site.

110. The method of claim 108, wherein the one or more primers are affinity tagged.

111. The method of claim 110, wherein the affinity tag is biotin.

112. The method of claim 100, wherein the step of isolating an ACE sequence associated with a chromatin-protein complex comprises treatment with a proteinase.

113. The method of claim 100, wherein the step of treating the cross-linked chromatin-protein complexes to reduce the size of the DNA sequences associated with the complexes comprises a treatment of sonication.

114. A library containing DNA sequences isolated according to the method of claim 100.

115. An array containing DNA sequences isolated according to the method of claim 100.

116. The method of claim 66 wherein the active chromatin elements are a fixed length.

117. The method of claim 116 wherein the active chromatin elements are monotagged.

118. The method of claim 117 wherein the active chromatin elements are direct monotagged.

119. The method of claim 117 wherein the active chromatin elements are indirect monotagged.

120. A method of preparing fixed length direct monotagged nucleic acids comprising:
a) treating genomic DNA with an agent that cleaves DNA;
b) ligating the treated genomic DNA with a blunt or T-tailed linker containing a type IIs restriction endonuclease restriction site; and
c) treating the ligated DNA with a type IIs restriction enzyme.

121. The method of claim 120 wherein step a) is performed using DNase I in the presence of manganese.

122. A method of preparing fixed length indirect monotagged nucleic acids comprising:
a) treating genomic DNA with an agent that cleaves DNA;
b) capturing the treated genomic DNA;
c) treating the captured genomic DNA with a restriction enzyme;
d) ligating the genomic DNA of step c) with a linker comprising a type IIs restriction enzyme site; and
e) treating the ligated DNA with a type II restriction enzyme.

123. The method of claim 122 wherein the agent that cleaves DNA is a restriction endonuclease.

124. The method of claim 122 wherein the cleavage sites within the genomic DNA are captured following biotinylation or ligation of a biotinylated linker.

125. A method of profiling of ACEs in a cell, comprising:
a) preparing genomic DNA according to the method of claim 120 or 122; and
b) hybridizing the genomic DNA to an array comprising active chromatin element.

126. A method of profiling a cell, comprising:
a) preparing genomic DNA according to the method of claim 120 or 122; and
b) hybridizing the genomic DNA to an array comprising a plurality of DNA sequences.
